(19) United States
(12) Patent Application Publication
(10) Pub. No.: US 2023/0404529 A1
(43) Pub. Date: Dec. 21, 2023

(54) APPARATUS FOR ULTRASOUND SCANNING

(71) Applicant: VEXEV PTY LTD, Surry Hills (AU)

(72) Inventors: Eamonn Stuart COLLEY, Alexandria (AU); John Eric CARROLL, Surry Hills (AU); Kyle James FORSTER, Northbridge (AU)

(21) Appl. No.: 18/462,188

(22) Filed: Sep. 6, 2023

Related U.S. Application Data
(63) Continuation of application No. PCT/AU2022/051385, filed on Nov. 18, 2022.

(30) Foreign Application Priority Data
Nov. 18, 2021 (AU) 2021903714

(51) Int. Cl. A61B 8/00

(52) U.S. Cl. CPC A61B 8/4281; A61B 8/587; A61B 8/4461

(57) ABSTRACT

Disclosed is a robotic ultrasound system comprising an ultrasound probe and a transport mechanism for moving the ultrasound probe in at least one direction. The robotic ultrasound system also comprises a scanning bed comprising the transport mechanism and the ultrasound probe, the scanning bed having a fluid filled portion for the ultrasound probe, wherein a fluid of the fluid filled portion allows transmission of ultrasound waves from the ultrasound probe to a surface of the robotic ultrasound system.

### CROSS-REFERENCE TO RELATED APPLICATIONS

The present application is a continuation of International Application No. PCT/AU2022/051385, filed Nov. 18, 2022, which claimed priority to Australian Application No. 2021903714, filed Nov. 18, 2021, the disclosures of which are hereby incorporated by reference in their entireties.

### TECHNICAL FIELD

The present invention relates to ultrasound scanning, and particularly to an apparatus for performing an automated ultrasound scan.

### BACKGROUND

Ultrasounds machines are commonly used in a range of medical procedures, such as diagnostic imaging. Typically, ultrasound scans are performed by trained operators using an ultrasound machine. The ultrasound machine has a number of different settings that are controlled by the operator and are configured before and during the course of the scan, with the settings being adjusted based on characteristics of a patient.

During the scan, the operator may press an ultrasound probe on to the skin of a patient. The probe typically has ultrasound gel applied to ensure contact with the skin and eliminate any air gaps between the probe and the skin. Maintaining contact between the probe and the skin of the patient ensures correct operation of the ultrasound system.

During the ultrasound scan the operator moves the ultrasound probe on the skin of the patient to provide a real-time 2D/3D view of the anatomic region of interest of the patient. Ultrasound systems are therefore generally built to process data collected from the scanner in real-time and to display the processed data on a screen to the operator to provide visual feedback. Allowing the operator to view the output of the probe allows the operator to quickly adjust location and settings of the probe, settings such as gain, focus, regions of interest (ROI), etc.

The display is generally rendered in a 2D slice view (B-mode) but shortcomings in interpreting 3D objects for two dimensional imaging have led to the development of 3D probes or 2D probes with attachments to create 3D volumes. However, the 3D probes and 2D probes with attachments require much higher processing power than a 2D probe, which leads to higher costs, poor fidelity in some cases, and limitations to the size of each acquired 3D volume.

It is the role of the ultrasound operator to infer the point of interest and to make or capture measurement to refer to a clinician requesting the data. Such an arrangement can lead to a high degree of subjectivity and inconsistency between cases, especially when cases have different ultrasound operators. It may also be difficult for sonographers to position their arm in certain locations, such as underneath, for prolonged periods of time, which can lead to muscle fatigue in the short term and injury in the long term.

### SUMMARY

This Summary is provided to introduce a selection of concepts in a simplified form that are further described below in the Detailed Description. This Summary is not intended to identify essential features of the claimed subject matter, nor is it intended to be used to limit the scope of the claimed subject matter.

One embodiment includes a robotic ultrasound system comprising: an ultrasound probe; a transport mechanism for moving the ultrasound probe in at least one direction; a scanning bed comprising the transport mechanism and the ultrasound probe, the scanning bed having a fluid filled portion for the ultrasound probe, wherein a fluid of the fluid filled portion allows transmission of ultrasound waves from the ultrasound probe to a surface of the robotic ultrasound system.

In one embodiment the scanning bed of the robotic ultrasound system further comprises: a housing; and a flexible portion on which an anatomic region of interest of a patient may be placed for scanning by the robotic ultrasound system, the flexible portion being in contact with the fluid of the fluid filled portion.

In one embodiment the robotic ultrasound system further comprises: a wall to support the flexible portion.

In one embodiment the wall is removable.

In one embodiment the robotic ultrasound system further comprises a level sensor to monitor a level of the fluid.

In one embodiment the level sensor is the ultrasound probe.

In one embodiment the level sensor is used to determine a presence of the anatomic region of interest on the flexible portion.

In one embodiment the ultrasound probe scans the anatomic region of interest from one side.

In one embodiment the flexible portion conforms to a shape of the anatomic region of interest.

In one embodiment the transport mechanism for moving the ultrasound probe is located in the fluid filled portion.

In one embodiment the transport mechanism for moving the ultrasound probe provides movement of the ultrasound probe in a direction selected from a set of directions comprising lateral, longitudinal, vertical, pitch, roll, and yaw.

In one embodiment the ultrasound probe is submerged in the fluid of the fluid filled portion.

In one embodiment the scanning bed of the robotic ultrasound system further comprises: a second ultrasound probe submerged in the fluid of the fluid filled portion; and a second transport mechanism for moving the second ultrasound probe in at least one direction.

In one embodiment the first and the second transport mechanisms travel on an inner and an outer rail.

In one embodiment the fluid is a mineral oil.

In one embodiment the fluid filled portion contains a calibration object for calibrating the ultrasound probe.

In one embodiment the fluid of the fluid filled portion is pressurised.

In one embodiment the fluid of the fluid filled portion is heated.

In one embodiment the fluid filled portion is heated to increase venous and arterial dilation.

In one embodiment a level of the fluid in the scanning bed may be varied.

In one embodiment the robotic ultrasound system further comprises a tower to which the scanning bed is attached.

In one embodiment the scanning bed is height adjustable by moving relative to the tower.

One embodiment includes a method of performing an ultrasound scan using the robotic ultrasound system, the method comprising: initiating a scan of the anatomic region of interest from a robotic ultrasound system controller; calibrating the robotic ultrasound system; and collecting data for a length of a scannable volume.

In one embodiment, calibrating the robotic ultrasound system comprises capturing scan data of the calibration object within the fluid filled portion.

### BRIEF DESCRIPTION OF FIGURES

At least one embodiment of the present invention is described, by way of example only, with reference to the accompanying figures.

FIG. 1 illustrates a functional block diagram of an example processing system that can be utilised to embody or give effect to a particular embodiment;

FIG. 2 illustrates an example network infrastructure that can be utilised to embody or give effect to a particular embodiment;

FIGS. 3A and 3B illustrates a robotic ultrasound system in accordance with one embodiment of the invention;

FIG. 4 illustrates an alternative view of the robotic ultrasound system of FIGS. 3A and 3B;

FIG. 5 illustrate a view of the robotic ultrasound system of FIGS. 3A and 3B during scanning of an arm;

FIG. 6A illustrates a view of the robotic ultrasound system of FIGS. 3A and 3B with a flexible portion of a shell removed;

FIG. 6B illustrates the robotic ultrasound system of FIGS. 3A and 3B with a leg for scanning;

FIG. 7 illustrates a robotic ultrasound system in accordance with one embodiment of the invention;

FIG. 8 illustrates the robotic ultrasound system of FIG. 7;

FIG. 9 illustrates a transport mechanism of a robotic ultrasound system in accordance with one embodiment of the invention;

FIG. 10 illustrates a carriage of the transport mechanism of FIG. 9;

FIG. 11 illustrates a dual probe transport of a transport mechanism of a robotic ultrasound system in accordance with one embodiment of the invention;

FIG. 12 illustrates an alternative view of the dual probe transport of FIG. 11;

FIG. 13 illustrates a method of robotic ultrasound system scanning in accordance to one embodiment of the invention;

FIG. 14 illustrates a block diagram of a robotic ultrasound system according to one embodiment of the invention;

FIG. 15 illustrates a portable robotic ultrasound system in accordance with one embodiment of the invention;

FIGS. 16A to 16C illustrate an alternative carriage in accordance with one embodiment of the invention;

FIGS. 17A to 17C illustrate the alternative carriage of FIGS. 16A to 16C within a housing of a robotic ultrasound system;

FIG. 18 illustrates a portable robotic ultrasound system in accordance with one embodiment of the invention;

FIGS. 19A and 19B illustrate internal of a scanning bed of the portable robotic ultrasound system of FIG. 18;

FIGS. 20A, 20B and 20C illustrate a transport mechanism of a robotic ultrasound system in accordance with one embodiment of the invention; and

FIGS. 21A and 21B illustrates an alternative dual probe transport of a transport mechanism of a robotic ultrasound system in accordance with one embodiment of the invention.

### DETAILED DESCRIPTION

The following modes, given by way of example only, are described in order to provide a more precise understanding of one or more embodiments. In the figures, like reference numerals are used to identify like parts throughout the figures.

Disclosed is robotic ultrasound system that can perform robotic tomographic ultrasound scanning and that can scan an anatomic region of interest (AROI) of a patient without the need for a skilled ultrasound operator to operate and conduct the ultrasound scan. The robotic ultrasound system, sometimes referred to as an ultrasound system, may comprise an ultrasound probe and also include a transport mechanism for moving the ultrasound probe in at least one direction. The ultrasound system may include a housing for the transport mechanism and ultrasound probe, the housing having a fluid filled portion for the ultrasound probe wherein a fluid of the fluid filled portion allows transmission of ultrasound waves from the ultrasound probe to a surface of the ultrasound system.

The disclosed robotic ultrasound system may comprise an ultrasound probe and a transport mechanism for moving the ultrasound probe in at least one direction. The robotic ultrasound system may also comprise a scanning bed comprising the transport mechanism and the ultrasound probe, the scanning bed having a fluid filled portion for the ultrasound probe, wherein a fluid of the fluid filled portion allows transmission of ultrasound waves from the ultrasound probe to a surface of the robotic ultrasound system.

One or more embodiments of the robotic ultrasound system are described, including a single ultrasound probe and a dual ultrasound probe configuration.

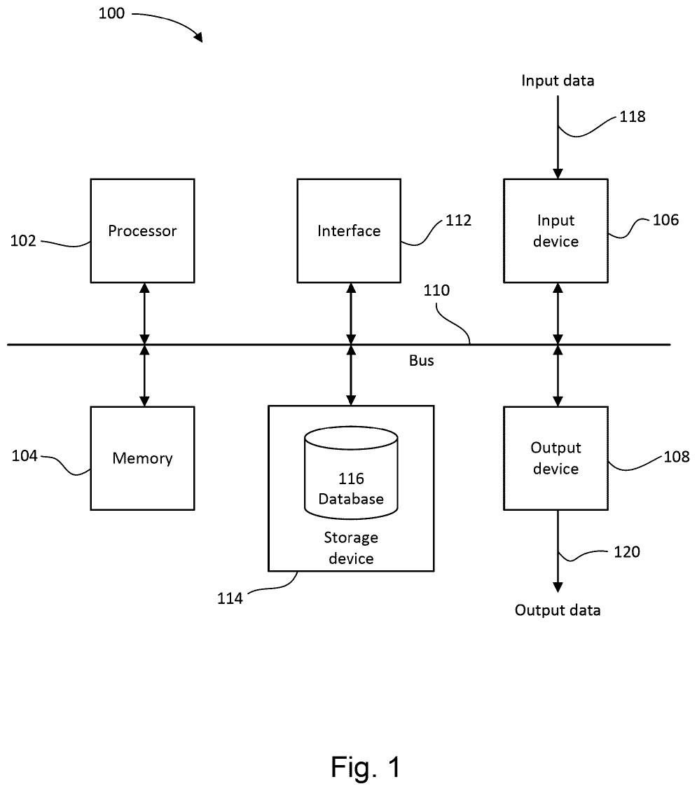

A particular embodiment of the present invention can be realised using a processing system, an example of which is shown in FIG. 1. In particular, the processing system 100 generally includes at least one processor 102, or processing unit or plurality of processors, memory 104, at least one input device 106 and at least one output device 108, coupled together via a bus or group of buses 110. In certain embodiments, input device 106 and output device 108 could be the same device. An interface 112 can also be provided for coupling the processing system 100 to one or more peripheral devices, for example interface 112 could be a PCI card, PCIe card or PC card. At least one storage device 114 which houses at least one database 116 can also be provided. The memory 104 can be any form of memory device, for example, volatile or non-volatile memory, solid state storage devices, magnetic devices, etc. The processor 102 could include more than one distinct processing device, for example to handle different functions within the processing system 100.

Input device 106 receives input data 118 and can include, for example, a keyboard, a pointer device such as a pen-like device or a mouse, audio receiving device for voice controlled activation such as a microphone, data receiver or antenna such as a modem or wireless data adaptor, data acquisition card, etc. Input data 118 could come from different sources, for example keyboard instructions in conjunction with data received via a network. Output device 108 produces or generates output data 120 and can include, for example, a display device or monitor in which case output data 120 is visual, a printer in which case output data 120 is printed, a port for example a USB port, a peripheral component adaptor, a data transmitter or antenna such as a modem or wireless network adaptor, etc. Output data 120 could be distinct and derived from different output devices, for example a visual display on a monitor in conjunction with data transmitted to a network. A user could view data output, or an interpretation of the data output, on, for example, a monitor or using a printer. The storage device 114 can be any form of data or information storage means, for example, volatile or non-volatile memory, solid state storage devices, magnetic devices, etc.

In use, the processing system 100 is adapted to allow data or information to be stored in and/or retrieved from, via wired or wireless communication means, the at least one database 116. The interface 112 may allow wired and/or wireless communication between the processing unit 102 and peripheral components that may serve a specialised purpose. The processor 102 receives instructions as input data 118 via input device 106 and can display processed results or other output to a user by utilising output device 108. More than one input device 106 and/or output device 108 can be provided. It should be appreciated that the processing system 100 may be any form of terminal, server, specialised hardware, or the like. In some instances, such as when a touch screen display is used, the output device 108 and the input device 106 may be the same device.

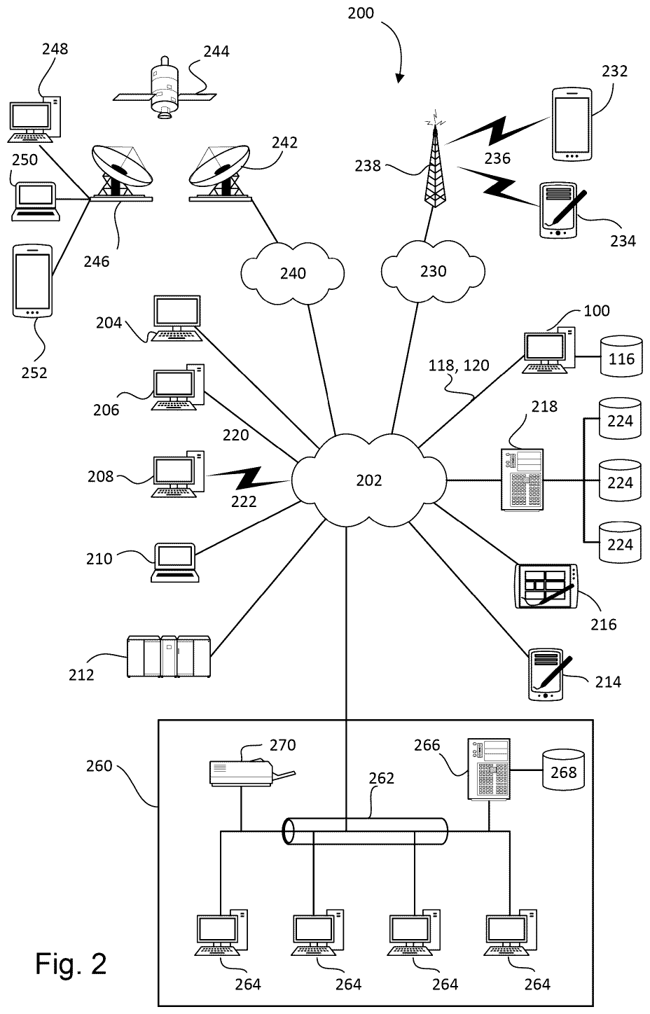

The processing system 100 may be a part of a networked communications system 200, as shown in FIG. 2. Processing system 100 could connect to network 202, for example the Internet or a WAN. Input data 118 and output data 120 could be communicated to other devices via network 202. Other terminals, for example, thin client 204, further processing systems 206 and 208, notebook computer 210, mainframe computer 212, PDA 214, pen-based computer or tablet 216, server 218, etc., can be connected to network 202. A large variety of other types of terminals or configurations could be utilised. The transfer of information and/or data over network 202 can be achieved using wired communications means 220 or wireless communications means 222. Server 218 can facilitate the transfer of data between network 202 and one or more databases 224. Server 218 and one or more databases 224 provide an example of an information source.

Other networks may communicate with network 202. For example, telecommunications network 230 could facilitate the transfer of data between network 202 and mobile, cellular telephone or smartphone 232 or a PDA-type device 234, by utilising wireless communication means 236 and receiving/transmitting station 238. Satellite communications network 240 could communicate with satellite signal receiver 242 which receives data signals from satellite 244 which in turn is in remote communication with satellite signal transmitter 246. Terminals, for example further processing system 248, notebook computer 250 or satellite telephone 252, can thereby communicate with network 202. A local network 260, which for example may be a private network, LAN, etc., may also be connected to network 202. For example, network 202 could be connected with ethernet 262 which connects terminals 264, server 266 which controls the transfer of data to and/or from database 268, and printer 270. Various other types of networks could be utilised.

The processing system 100 is adapted to communicate with other terminals, for example further processing systems 206, 208, by sending and receiving data, 118, 120, to and from the network 202, thereby facilitating possible communication with other components of the networked communications system 200.

Thus, for example, the networks 202, 230, 240 may form part of, or be connected to, the Internet, in which case, the terminals 206, 212, 218, for example, may be web servers, Internet terminals or the like. The networks 202, 230, 240, 260 may be or form part of other communication networks, such as LAN, WAN, ethernet, token ring, FDDI ring, star, etc., networks, or mobile telephone networks, such as GSM, CDMA, 4G, 5G etc., networks, and may be wholly or partially wired, including for example optical fibre, or wireless networks, depending on a particular implementation.

### Robotic Ultrasound System

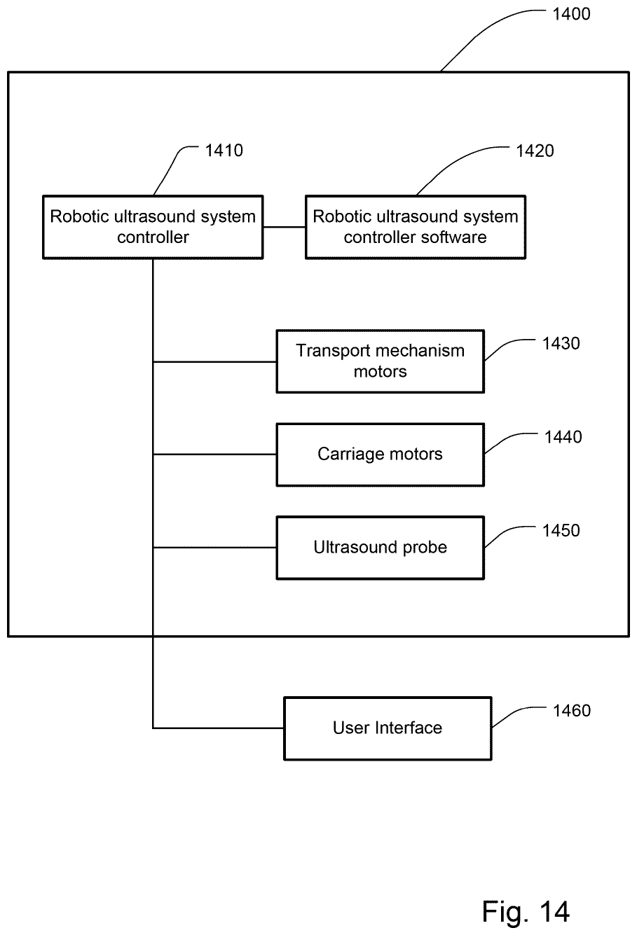

A configuration of a robotic ultrasound system 1400 will now be described in relation to FIG. 14. The robotic ultrasound system 1400 has a robotic ultrasound system controller 1410 that may be a computer, such as the processing system 100 described above. Alternatively, the robotic ultrasound system controller 1410 may be configured as two or more processing systems, such as the processing system 100 that communicate over a network such as the network 202. In one example, the robotic ultrasound system controller 1410 may be a computer system that communicates with one or more single board microcontrollers. While the robotic ultrasound system controller 1410 is shown as being within the robotic ultrasound system 1400, the robotic ultrasound system controller 1410 may also be located outside the robotic ultrasound system 1400 and signals sent to components within the robotic ultrasound system 1400. Robotic ultrasound system controller software 1420 is executed on the hardware of the robotic ultrasound system controller 1410 to perform the operations of the robotic ultrasound system 1400. The robotic ultrasound system controller 1410 is connected to components such as transport mechanism motors 1430 to control the operation of a transport mechanism, as well as carriage motors 1440 to move an ultrasound probe 1450 attached to a carriage of the transport mechanism motors 1430.

The robotic ultrasound system 1400 also has a user interface 1460 for communicating information to a user. The robotic ultrasound system controller 1410 may be connected to a display device, such a computer monitor, or liquid crystal display, to provide status information. The display device may also operate as a touch screen. The display may also provide information to allow the user to conduct manual operation and may render 3D vascular geometry for on-demand inspection by the operator. The status information shown may include scan in progress, scan complete with valid data or scan failed to indicate that the scan needs to be repeated. Status information may also be shown using LED indicators located on a region of the robotic ultrasound system 1400. Status information may alternatively, or additionally, be indicated using audible status updates from speakers of the robotic ultrasound system 14. The audible status may be a pre-recorded vocal statement or other audible forms. Status information may also be accessible over a network using a suitable app or web browser running on a computer connected, via a network connection, to the robotic ultrasound system controller 1410.

Example of a robotic ultrasound system will be described below. Described is an ultrasound imaging robot that may control the position and movement of an ultrasound probe as well as the generating and receiving of ultrasound waves. Use of the robotic ultrasound system may allow for simplified and fast, operator-independent, ultrasound imaging. The robotic ultrasound system moves the ultrasound probe within a sealed volume containing a fluid, such as mineral oil, with the ultrasound waves generated by the probe carried through the fluid before passing through the flexible portion of an outer shell of the robotic ultrasound system. The ultrasound probe is submerged in fluid of the fluid portion inside the flexible portion. The fluid is in contact with the flexible portion. A relevant part of the patient, an anatomic region of interest such as a limb, is positioned resting on an outside of a flexible portion of the shell. The ultrasound waves are transmitted through and conducted by the fluid and reflect from the patient, back through the flexible portion and fluid where the ultrasound waves are received by the ultrasound probe. The fluid under the flexible portion creates a hydrostatic pressure on the flexible portion as to the robotic ultrasound system is filled up to, or even above, the level of the flexible portion. In one example, the fluid may be heated for comfort of a patient when they are in contact with the flexible portion. The fluid may be heated by a heating element, located in the sealed volume, to control the temperature of the fluid. An additional heater may also be located in the fluid filled portion and/or an external reservoir that holds additional fluid.

The flexible portion is made of a flexible material such as thermoplastic polyurethane (TPU) and provides a suitable interface between the ultrasound probe and biological tissue of the patient. A thickness of the flexible material is chosen that balances strength against flexibility, as a thicker material may be less flexible but stronger, while thinner material may be more flexible but weaker. An example of a suitable thickness of the flexible material is 0.1 mm. In one embodiment the thickness of the material may vary, for example the side wall may be thicker than other areas of the flexible material. The flexible portion may have a contoured shape that allows an anatomic region of interest to be placed on the flexible portion and be in contact with the flexible portion to allow scanning by the ultrasound probe. The flexible portion material may have ultrasound conductive properties and be able to be formed without air pockets as air pockets would reduce conductivity of the ultrasound. Preferably, the flexible portion material does not compress or deform the tissue or vasculature being scanned. If the flexible portion material is made of material with elastic properties then the flexible portion material may allow freedom for the anatomic region of interest of the patient to sink in with the flexible region conforming to a shape of the anatomic region of interest due to the hydrostatic pressure from the fluid. The flexible portion provides a region for the patient to hold or place the anatomic region of interest, e.g. a body segment such as an arm or a leg, in a position. The shape of the flexible portion may encourage the patient to place the body part in approximately the same position each time the robotic ultrasound system is used. As the patient is in contact with the flexible portion, the flexible portion material may be required to withstand disinfecting processes.

During operation of the robotic ultrasound system it is important that there are no air gaps between the ultrasound probe and the patient as the air gaps may interfere with ultrasound transmission. The fluid in the fluid filled portion may prevent air gaps between the ultrasound probe and the flexible material, while application of ultrasound, aqueous gel to the anatomic region of interest before being placed on the flexible portion may prevent air gaps between the flexible material and the anatomic region of interest.

The ultrasound probe, also referred to as an ultrasound transducer, is movable within the robotic ultrasound system to allow the ultrasound probe to be positioned to scan an anatomic region of interest. In one example, the ultrasound probe is an Interson SP-LO1 probe which houses an ultrasound transducer, although other probes may be used and the probe does not have to be a hand held design. Data and power for the ultrasound probe may be provided via a USB or a micro-coaxial cable, where to data may include communication for control signals and data between a robotic ultrasound system controller, such as the robotic ultrasound system controller 1410 described above or other controller. A beamformer is also used to generate the send and receive of the ultrasound transducer, which is used to create ultrasound images. The beamformer may be part of the ultrasound probe or provided over the USB or micro-coaxial cable or an additional cable to the ultrasound probe. The ultrasound probe may sit in a cradle, located on a carriage that moves on rails. The cradle may be exchanged with other cradles to allow the robotic ultrasound system to operate with different ultrasound probes where each cradle can be designed for mounting a different ultrasound probe, such different ultrasound probes made by different manufacturers. Changing between ultrasound probes may be done by swapping the cradle of the carriage and selecting a new ultrasound probe type in the software of the robotic ultrasound system controller.

The robotic ultrasound system controller may communicate, via the network 202, to a local or remote, e.g. cloud based, server by sending data collected from the ultrasound probe to the cloud. The collected data may be raw data from the ultrasound probe or may be processed in either the ultrasound probe or by the robotic ultrasound system controller software before being transmitted to the local or remote server. In one example of the robotic ultrasound system the ultrasound probe data may be customised before being uploaded to the remote server where the data can be accessed by clinicians for analysis and diagnosis.

The robotic ultrasound system controller may also provide the ability to enter patient information relating to the scan. The patient information may be entered before, during or after the scan. Alternatively, a patient may have an associated bar code scanned by a bar code reader. Alternatively, a two dimensional barcode may be used such as a QR code. A physical token can also be used, such as a near field communications tag that is read by a near field antenna connected to the robotic ultrasound system controller.

Self-calibration of the robotic ultrasound system may be carried out when the system is first powered or on demand when selected by a user, via the user interface. The self-calibration may be performed by imaging calibration objects, or phantoms, located within the fluid of the robotic ultrasound system. Each ultrasound probe in the robotic ultrasound system may be positioned to image a known calibration object and the image/data sent to the robotic ultrasound system controller where the scan of the known calibration objection is compared to an expected result and a calibration configuration is determined for the ultrasound probe, the robotic ultrasound system controller software or both. The calibration objects may be located in the fluid, the flexible portion or another part of the robotic ultrasound system that can be imaged by the ultrasound probe.

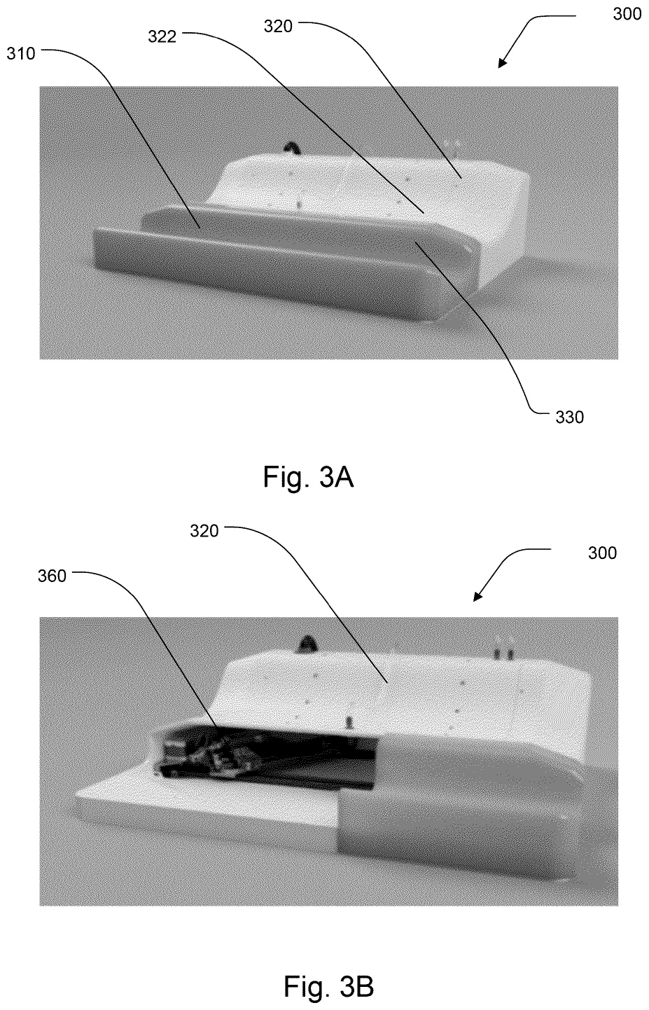

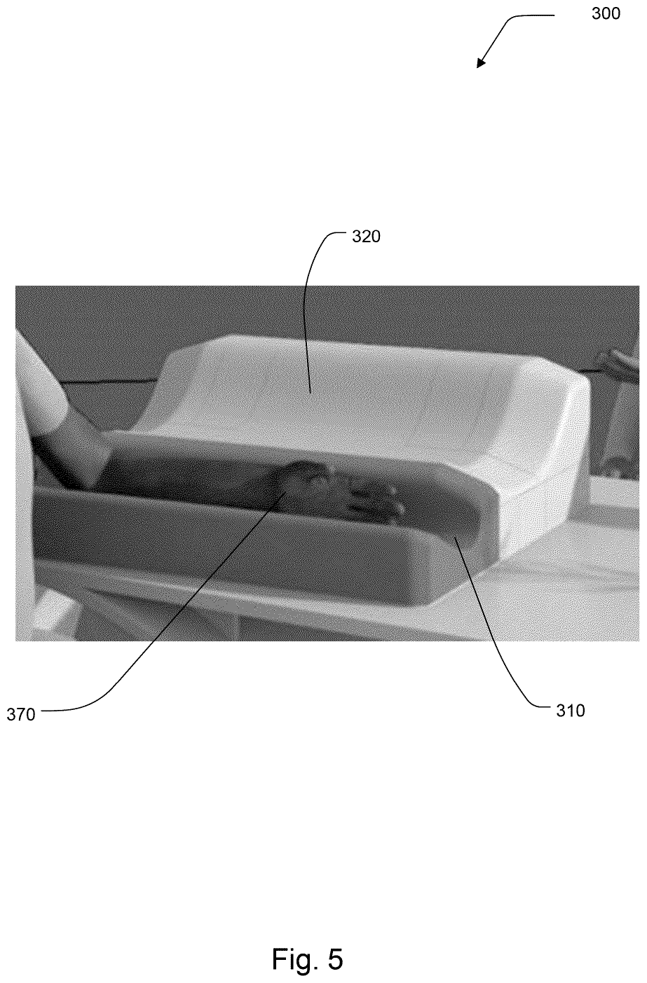

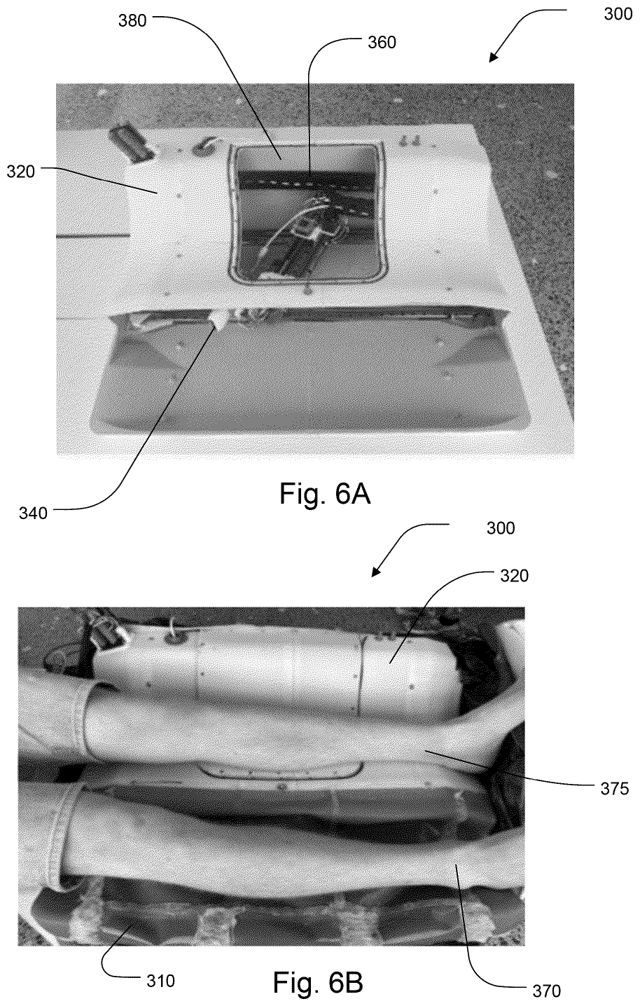

FIGS. 3A to 6 show a robotic ultrasound system 300 which comprises a scanning bed having two main parts, a flexible portion 310 and a housing 320. The flexible portion 310 may have a contoured portion for placing an anatomic region of interest of a patient. Within the flexible portion 310 and the housing 320 is a fluid portion. The fluid portion is in contact with the flexible portion 310. FIG. 3A shows the robotic ultrasound system 300 in an operation state, while FIG. 3B shows the robotic ultrasound system 300 with a cut away section of the flexible portion 310 and FIG. 6A shows the robotic ultrasound system 300 with the flexible portion 310 removed. As will be described in greater detail below, the robotic ultrasound system 300 operates an ultrasound probe in an automated or semi-automated manner that does not require a user of the robotic ultrasound system 300 to physically manipulate an ultrasound probe. Instead, the ultrasound probe of the robotic ultrasound system 300 moves by electromechanical means.

The housing 320 is typically made of a rigid material that is non-reactive to any fluid contained within the robotic ultrasound system 300. The housing 320 has a contoured region 322 that may be used as a resting place for a limb, or a non-scanned anatomic region of a patient during operation. An example may be seen in FIG. 6B where a scanned limb 370, or an anatomic region of interest, is located on the flexible portion 310 and a limb 375 is located on the housing 320. Such an arrangement allows for the patient to be in a comfortable position when scanning an inner region of the scanned limb 370.

As seen in FIG. 6A the housing 320 has an access opening 380 that provides access to a transport mechanism 360 inside the robotic ultrasound system 300. The transport mechanism 360 is used by the robotic ultrasound system to move an ultrasound probe 340 during a scanning process. The transport mechanism 360 will be described in more detail below. The access opening 380 will typically be held in place by fasteners and may have a seal to prevent the fluid within the robotic ultrasound system 300 from leaking.

The flexible portion 310 has a scooped, or concave, shape suitable for placing a anatomic region of interest, such as scanned limb 370. The flexible portion 310 is made from a flexible material that is resistant to the fluid within the robotic ultrasound system 300. A scan window 330 is a region of the flexible portion 310 where the ultrasound waves from a probe 340 pass through the flexible portion 310 to allow scanning of a limb of a patient. The size of the scan window 330 may determine a range of scanning by the probe 340 and also take into account possible movement of the probe 340 by the transport mechanism 360.

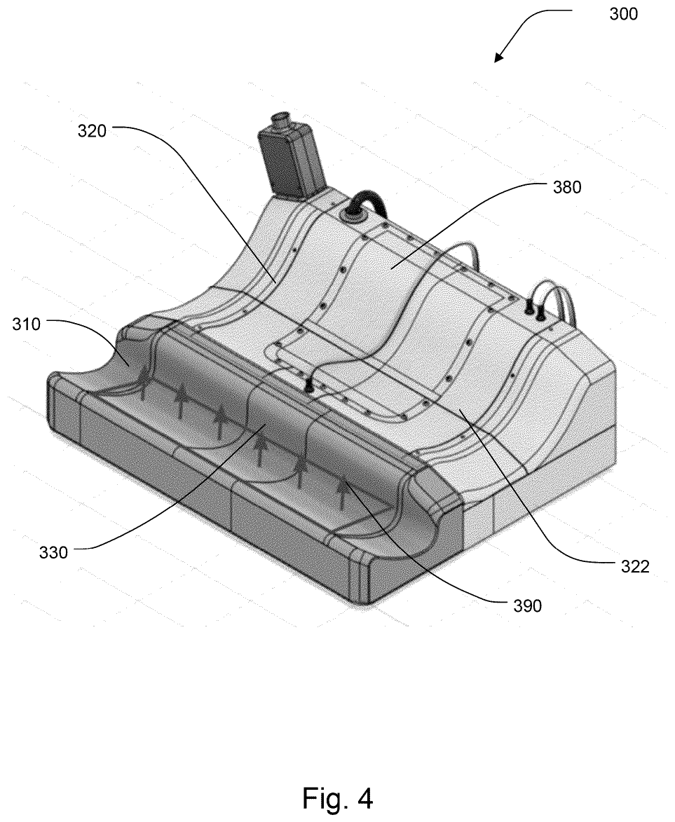

FIG. 4 shows hydrostatic pressure force 390 caused by the fluid in the robotic ultrasound system 300. The hydrostatic pressure force 390 will press on the anatomic region of interest located on the flexible portion 310 and allow the flexible portion 310 to conform to, and around, the anatomic region of interest. Alternatively, the fluid may be pumped in and out of the fluid filled portion of the robotic ultrasound system 300 and pressurised to vary the hydrostatic pressure force 390. For example, the hydrostatic pressure force 390 may be varied based on a shape of the region of interest to be scanned or to position the region of interest. The hydrostatic pressure force 390 may be varied to move the region of interest up and down on the flexible portion 310, with the hydrostatic pressure force 390 being increased to raise the region of interest and a lower hydrostatic pressure force 390 lowering the region of interest. In one example, the region of interest may be held in place and fluid pumped into the robotic ultrasound system 300. The flexible portion 310 may then form around the region of interest under the hydrostatic pressure force 390.

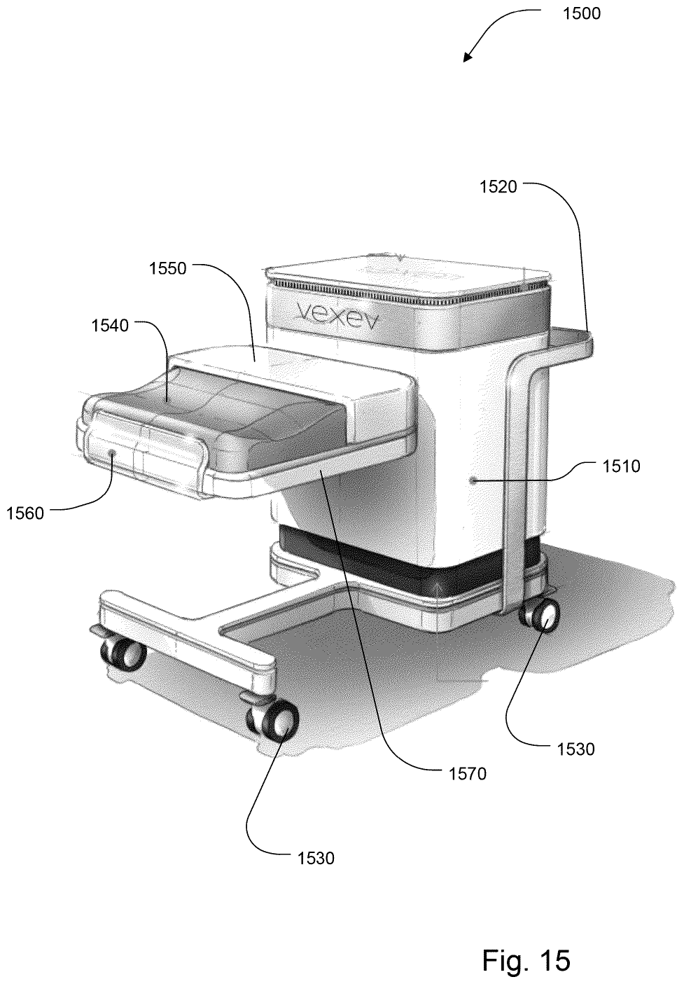

A portable robotic ultrasound system 1500 will be described in relation to FIG. 15. The portable robotic ultrasound system 1500 has a tower 1510 and a handle 1520 for moving the portable robotic ultrasound system 1500 around on wheels 1530. An adjustable scanning bed 1570 having a flexible portion 1540, located next to a housing 1550 and is mounted on the tower 1510. The flexible portion 1540 and the housing 1550 of the portable robotic ultrasound system 1500 may be configured in a similar manner to the robotic ultrasound system 300 described above.

The portable robotic ultrasound system 1500 has the adjustable scanning bed 1570 which has a removable fluid support wall 1560. The wall 1560 provides support for the flexible portion 1540 and may support the flexible portion 1540 and prevent or limit deformation the flexible portion 1540 caused by hydrostatic pressure of fluid in the portable robotic ultrasound system 1500. The fluid support wall 1560 prevents the flexible portion 1540 from spilling over a front edge of the portable robotic ultrasound system 1500. The fluid support wall 1560 may also help the flexible portion 1540 conform to the region of interest and prevent overspill of the material of the flexible portion 1540. When the anatomic region of interest is placed on the flexible portion 1540, suitable connection between the material of the flexible portion 1540 and the region of interest can ensure transmission of the ultrasound waves. The fluid support wall 1560 may be removable, allowing removal for scans when the fluid support wall 1560 may interfere or limit positioning of the region of interest for scanning. The fluid support wall 1560 may be used on the described robotic ultrasound systems including the robotic ultrasound system 300 described above.

The tower 1510 contains a pumping system to pump fluid from a reservoir, located in the tower 1510, to the adjustable scanning bed 1570. The scanning bed may be adjusted for height or rotated to adjust the angle and is moved by gearing driven by motors in the tower 1510. The height and rotation of the scanning bed may be changed, relative the tower 1510. The tower 1510 also houses electrical components, such as a computer or micro-controllers and an ultrasound beamformer. The reservoir holds fluid and may also have a heater to keep the fluid in the reservoir at a set temperature. The portable robotic ultrasound system 1500 may also have a touchscreen monitor to provide a user interface for controlling operation of the system, as well as for displaying status information. While the portable robotic ultrasound system 1500 shows an adjustable scanning bed 1570 attached to the tower 1510, the adjustable scanning bed 1570 may be replaced with other attachments.

### Alternative Robotic Ultrasound System

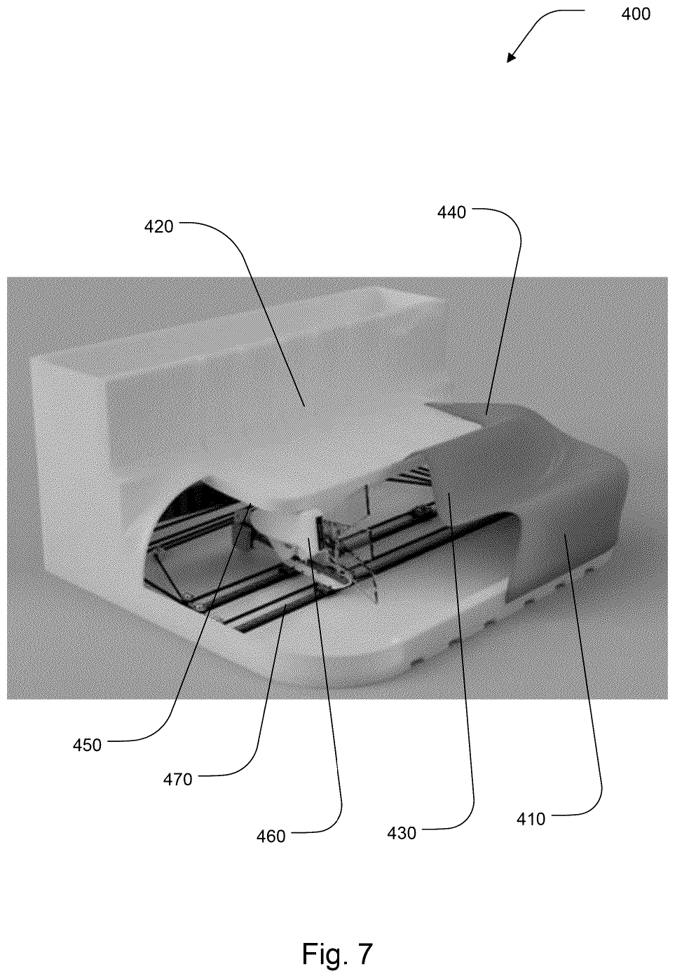

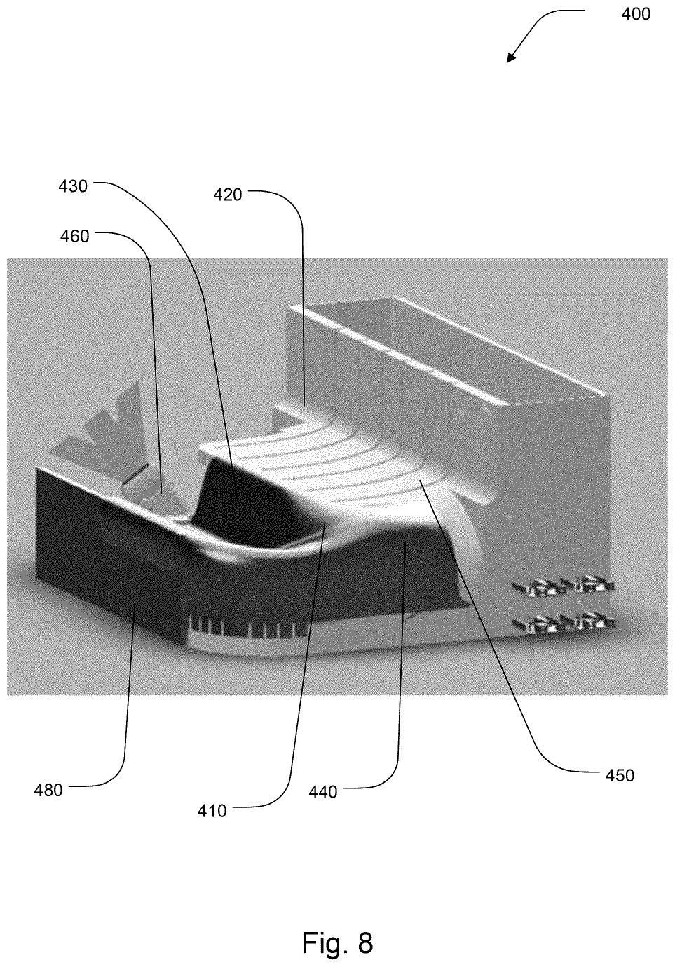

FIG. 7 and FIG. 8 show an alternative robotic ultrasound system. Shown is a robotic ultrasound system 400 which comprises a scanning bed with a contoured flexible portion 410 and a housing 420. The robotic ultrasound system 400 also has a scan window 430, similar to the robotic ultrasound system 300 described above. Both FIG. 7 and FIG. 8 show the robotic ultrasound system 400 with a section of the flexible portion 410 removed to show internals of the robotic ultrasound system. As with the robotic ultrasound system 300, the robotic ultrasound system 400 also includes a probe 460 and a transport mechanism 470 for movement of the probe 460.

The robotic ultrasound system 400 includes a curved flexible portion 440 to allow the probe 460 access to regions of the anatomic region of interest of a patient that may be difficult to access using the robotic ultrasound system 300. The flexible portion curvature 440 has both a contour, like the flexible portion 310 and the flexible portion 410, as well as a curvature. That is, the flexible portion curvature 440 may allow scanning of non-straight anatomic region of interest of the patient. One example is scanning an arm, shoulder, axilla or elbow. Another example may be scanning a leg, such as groin, knee or foot. In the example of scanning a foot, the patient may have a leg on the flexible portion 410 and the top of the foot extending around the curved flexible portion 440. The curve of the curved flexible portion 440 allows the probe 460 to scan a curved anatomic region of interest such as a shin, ankle and top of the foot region without the patient being reposition between scans. A fluid support wall 480, similar to the detachable fluid support wall 1560 may also be included in the robotic ultrasound system 400. The robotic ultrasound system 400 may be placed on a height adjustable platform or trolley to allow height and rotation adjustment to allow the patient to be comfortable during scanning.

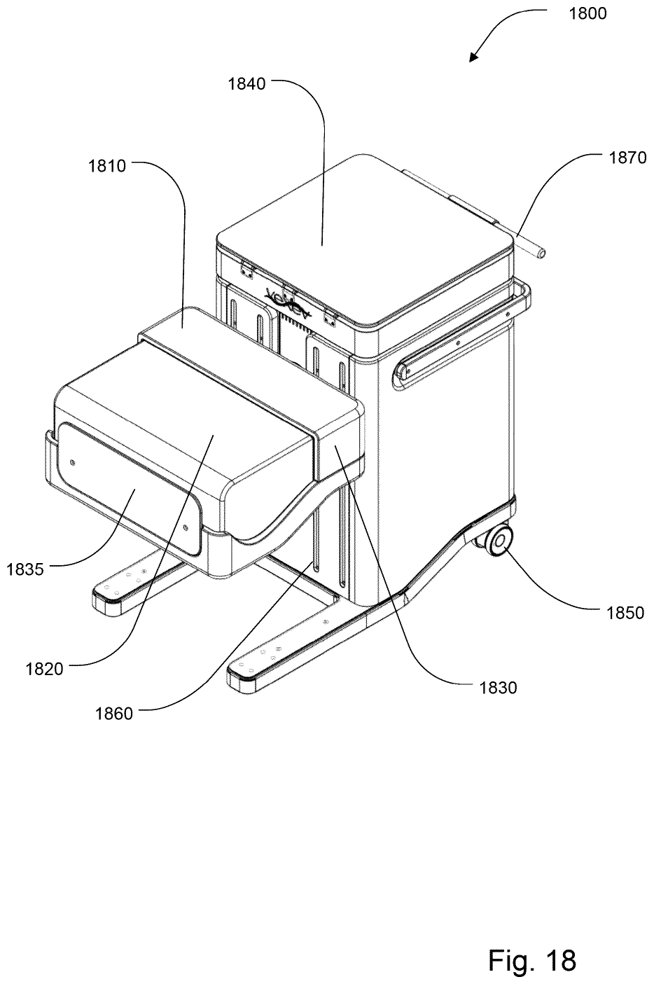

Another alternative portable robotic ultrasound system 1800 will be described in relation to FIG. 18. The portable robotic ultrasound system 1800 has two main components, a tower 1840 and, connected to the tower 1840, an adjustable scanning bed 1810. The adjustable scanning bed 1810 may be rotated as well adjusted for height using tracks 1860. The adjustable scanning bed 1810 has a flexible portion 1820 that has a different shape to the flexible portion 310. Where the flexible portion 310 has a contoured shape, the flexible portion 1820 is flat, the flexible portion 1820 is featureless, non-rigid, and conforms to the shapes imposed by a load caused by the anatomic region of interest. The flexible portion 1820 has a large area to allow the flexible portion 1820 to conform to the anatomic region of interest. In one embodiment, the flexible portion 1820 may have additional material that can wrap around the anatomic region of interest. When an anatomic region of interest is placed on the flexible portion 1820, for scanning, the region of interest may be wrapped in the flexible portion 1820 as the flexible material deforms under the weight of the region of interest. The deformation of the flexible portion 1820 allows the material to wrap the anatomic region of interest and allows the ultrasound to scan the anatomic region of interest from multiple angles. The adjustable scanning bed 1810 has a housing 1830 as well as a fluid support wall 1835 that supports the weight of the fluid and the flexible portion 1820.

A side of the flexible portion 1820 may allow scanning of a side of a patient, torso, or armpit through a side scanning window 1824. The region of the patient to be scanned is placed on the side scanning window 1824 and the ultrasound probe directed to scan the area.

The tower 1840 contains items such a power supply, which may include an uninterruptable power supply, a beamformer, motor microcontroller, fluid reservoir, fluid pump, an electrocardiogram, motors for moving the adjustable scanning bed 1810, an ultrasound beamformer and microcontrollers. The adjustable scanning bed 1810 may have at least one carriage, an ultrasound probe, fluid heater and a fluid level sensor. On the outside of the tower 1840 is a detachable touch screen controller, wheels 1850 and a handle 1870.

### Single Probe Transport Mechanism for a Robotic Ultrasound System

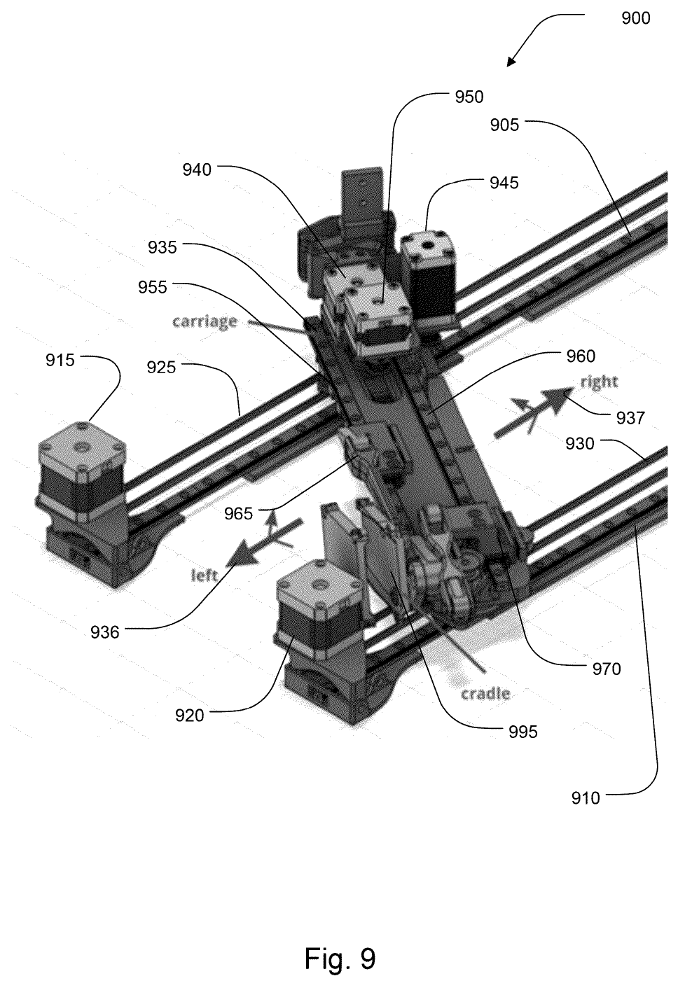

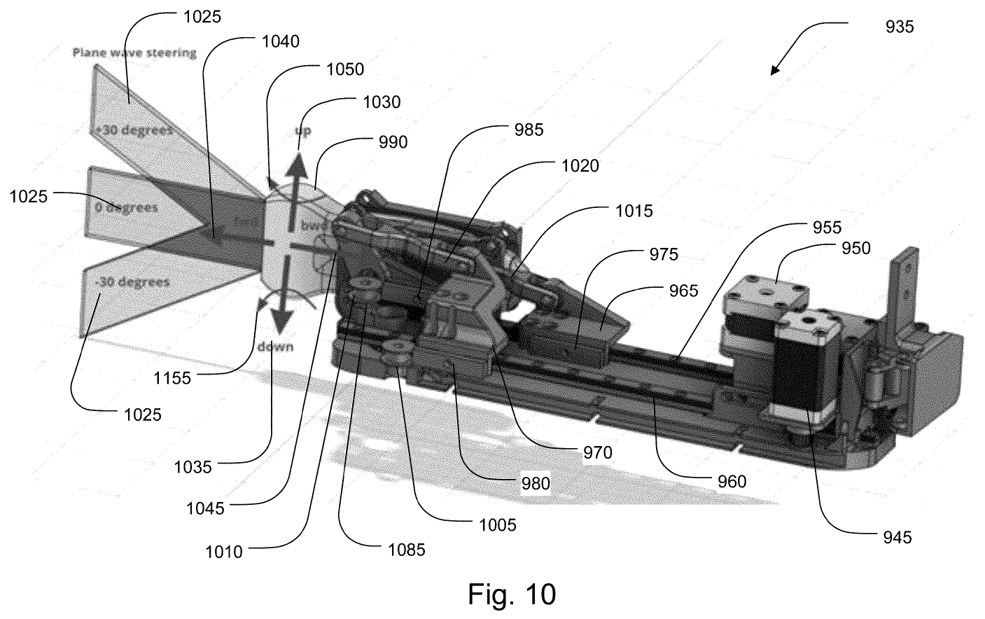

A transport mechanism for a single ultrasound probe will now be described in relation to FIG. 9, which shows a transport mechanism 900, and FIG. 10 which shows a carriage 935. The transport mechanism 900 may be located in the fluid filled portion of the robotic ultrasound system. The transport mechanism 900 has an outer rail 905 located further away from an anatomic region of interest of a patient than an inner rail 910. The carriage 935 is mounted on the outer rail 905 and the inner rail 910 and driven by an outer rail motor 915 and an inner rail motor 920 respectively. The carriage 935 connects to the outer rail motor 915 and the inner rail motor 920 using an outer rail belt 925 and an inner rail belt 930. The outer rail motor 915 and the inner rail motor 920 drive the carriage 935 independently of each other to control yaw, also referred to as rotation or slew, of the carriage 935. For example, yaw of the carriage 935 may be controlled by moving the outer rail motor 915 and the inner rail motor 920 separately at different speeds or in different directions. That is, using the outer rail motor 915 to drive an outer rail connection of the carriage 935 in a left direction 936, while keeping the inner rail motor 920 stationary, will result in the carriage 935 rotating clockwise when viewed in FIG. 9. The carriage 935 will also rotate clockwise if the inner rail motor 920 drives an inner rail connection of the carriage 935 in a right direction 937 while the outer rail motor 915 is stationary. Clockwise rotation is also possible if the outer rail motor 915 drives the outer rail connection in the left direction 936 and the inner rail motor 920 drives the inner rail connection in the right direction 937. The carriage 935 may be rotated in an anticlockwise direction by moving the outer and the inner rail connection in the opposite direction to those described above.

While yaw of the carriage 935 is controlled by moving the carriage 935 along the outer rail motor 915 and the inner rail motor 920, additional movement of an ultrasound probe 990, carried in a cradle 995, is also possible. The additional movement of the ultrasound probe 990 is achieved by moving the cradle 995 on the carriage 935. The carriage based motion allows the ultrasound probe 990 to pitch up and down, raise and lower as well as move forwards and backwards where forwards moves the ultrasound probe 990 toward the scanning window and the anatomic region of interest and backwards moves the ultrasound probe 990 away from the scanning window. The motion of the transport mechanism 900 and the carriage 935 allows for adjustment of the pitch, height and distance to the anatomic region of interest of the ultrasound probe 990. The ultrasound probe 990 is submerged in the fluid of the fluid filled portion of the robotic ultrasound system.

The carriage based motion of the ultrasound probe 990 is driven by a left rail motor 940, a right rail motor 945 and a middle motor 950. The left rail motor 940 drives a left rail carriage slide 965 on a left rail 955 using a belt, not shown, that returns via a sprocket. The belt is attached to a left rail belt attachment point 975. Movement of the left rail carriage slide 965 manipulates a left rail lever arm 1015. The right rail motor 945 drives a right rail carriage slide 970 on a right rail 960 using a belt, not shown, that returns via a right rail sprocket 1005. The belt is attached to a right rail belt attachment point 980. Movement of the right rail carriage slide 970 manipulates a right rail lever arm 1020. The middle motor 950 does not have an associated rail on the carriage 935. Instead, a belt, not shown, for the middle motor 950 returns via a middle sprocket 1010 and is attached to a middle belt attachment point 985.

The carriage based motion of the ultrasound probe 990 allows the ultrasound probe 990 to move up 1030 and down 1035, forward 1040 and backwards 1045, as well as pitch up 1050 and pitch down 1055. The motion is achieved by movement of three levers, the left rail lever arm 1015, the right rail lever arm 1020 and a middle lever arm. Motion of the three levers is co-ordinated by the robotic ultrasound system controller, such as the robotic ultrasound system controller 1410, executing the robotic ultrasound system controller software. For example, moving all three levers in the forward 1040 direction at the same speed will move the ultrasound probe 990 forwards. Moving the motors to different positions will allow the ultrasound probe 990 to move up 1030, down 1035, pitch up 1050 and pitch down 1055.

Operation of the outer rail 905 and the inner rail 910, along with movement of the carriage 935 provide movement in a lateral direction for the ultrasound probe 990. The lateral movement allows the ultrasound probe 990 to scan along the anatomic region of interest.

### Alternative Single Probe Transport Mechanism for a Robotic Ultrasound System

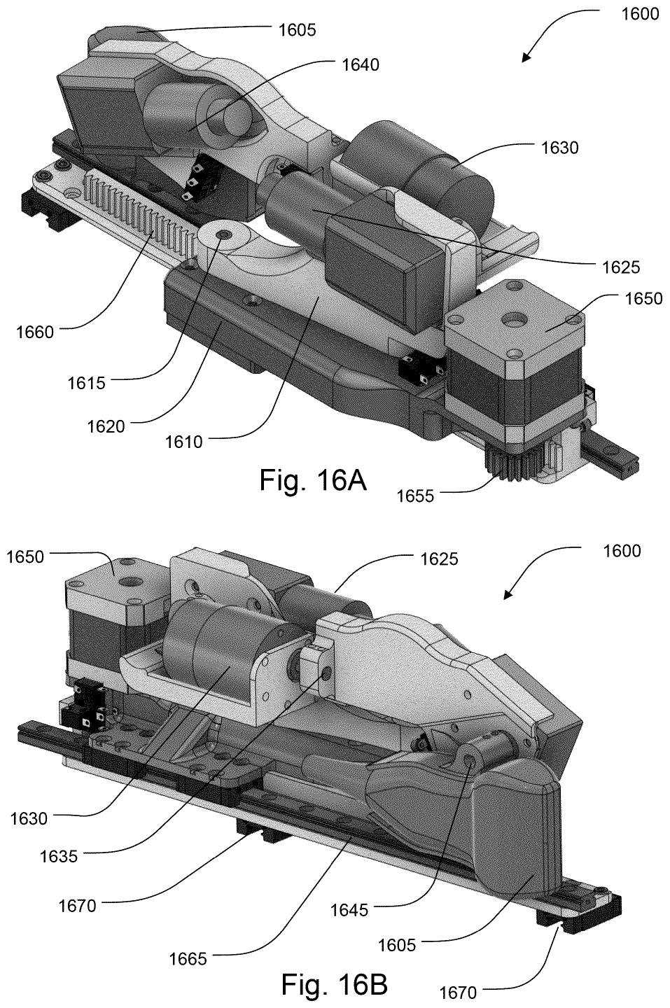

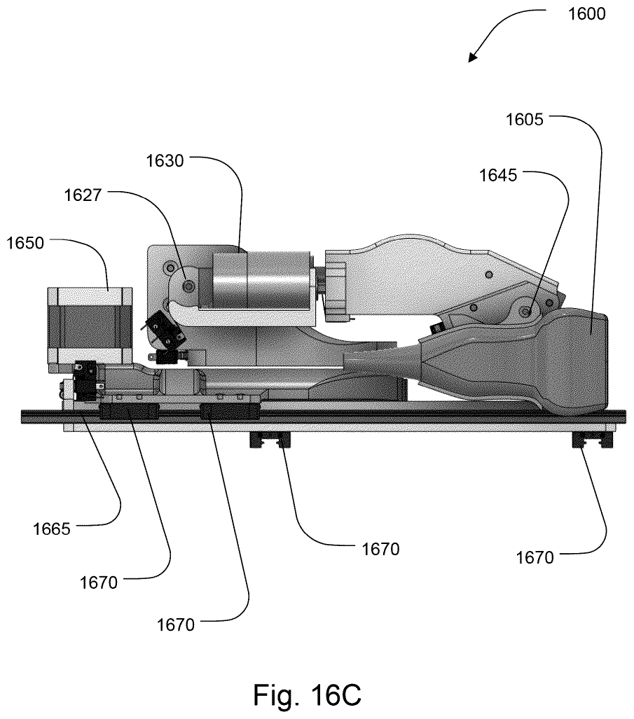

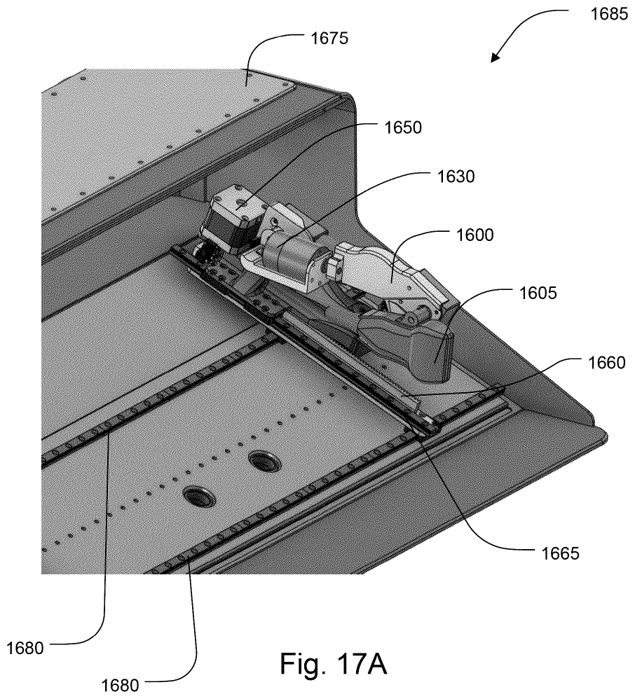

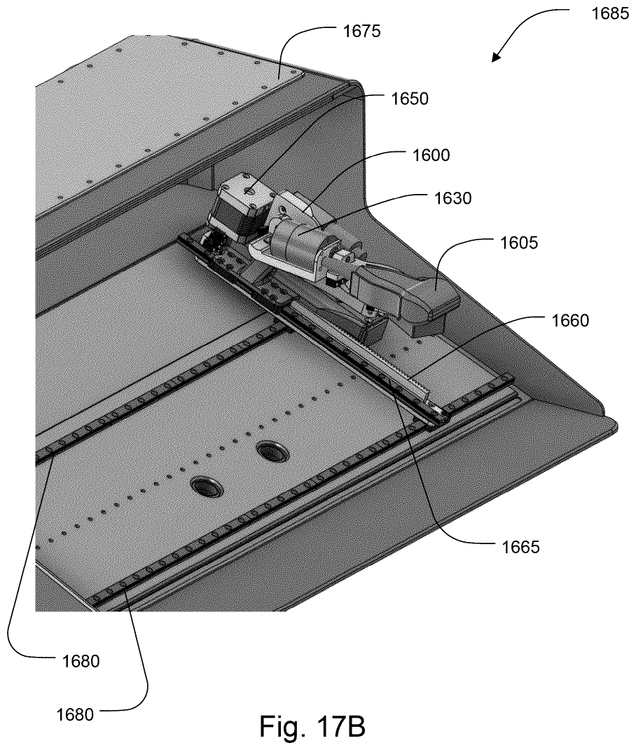

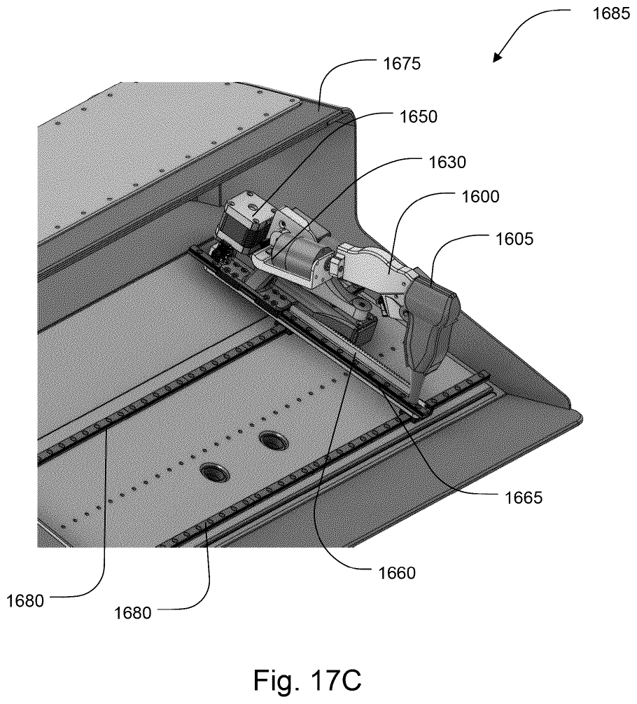

FIGS. 16A, 16B, 16C, 17A, 17B and 17C show an alternative transport mechanism for a single ultrasound probe. The mechanism is similar to the transport mechanism 900 described above and rides on a system of rails as described in relation to the transport mechanism 900. The alternative transport mechanism adds an additional degree of freedom with respect to an addition of a rolling axis on the arm holding a ultrasound probe.

The alternative transport mechanism has a carriage 1600 for moving an ultrasound cradle that holds an ultrasound probe 1605. The carriage 1600 has a plurality of components including a secondary carriage 1610, that rides on top of the carriage 1600, that may move back and forth to allow longitudinal translational movement of the ultrasound probe 1605. A part of the secondary carriage 1610 may rotate about a secondary carriage yaw rotation point 1615 which allows for rotational movement of the secondary carriage 1610 in yaw. The movement of the ultrasound probe 1605 about the secondary carriage yaw rotation point 1615 is driven by a yaw motor 1620, through a gearbox. A pitch motor 1625 is connected to the secondary carriage 1610, allowing up and down vertical movement of the ultrasound probe 1605 about a pitch rotation point 1627. Roll motion of the ultrasound probe 1605 may be achieved using a roll motor 1630 on the secondary carriage 1610 to rotate about a roll rotation point 1635. Pitch motion of the ultrasound probe 1605 may be achieved by an ultrasound probe rotation motor 1640 at the end of the secondary carriage 1610. The ultrasound probe rotation motor 1640 may rotate the ultrasound cradle with the ultrasound probe 1605 about an ultrasound probe rotation point 1645. A longitudinal translational motor 1650 may drive a pinion 1655 along a rack 1660 to move the secondary carriage 1610 in a longitudinal direction, similar to movement forward 1040 and backwards 1045 described above. The secondary carriage 1610 moves in the longitudinal direction along a secondary carriage rail 1665. Linear bearings 1662 may attach the secondary carriage 1610 to the secondary carriage rail 1665.

FIGS. 17A, 17B and 17C show the carriage 1600 inside a robotic ultrasound system 1685. The robotic ultrasound system 1685 is shown with a partial housing 1675 as well as rails 1680 for the carriage 1600 to move along in a lateral direction, equivalent to the left direction 936 and the right direction 937 described above. The carriage 1600 connects to the rails 1680 with linear bearings 1670. As with the transport mechanism 900, the carriage 1600 may be driven along the rails 1680 using belts or other mechanisms. FIGS. 17A to 17C shows the ultrasound probe 1605 in three different positions. FIG. 17A shows the ultrasound probe 1605 directed horizontally, with a vertical orientation. FIG. 17B shows the ultrasound probe 1605 directed horizontally and rotated 90 degrees to have a horizontal orientation. FIG. 17C shows the ultrasound probe 1605 facing upwards, directed vertically. While FIGS. 17A to 17C show possible movement of the ultrasound probe 1605, the carriage 1600 provides for controlled movement of the ultrasound probe 1605 with six degrees of freedom, being lateral, longitudinal, vertical, pitch, roll, and yaw.

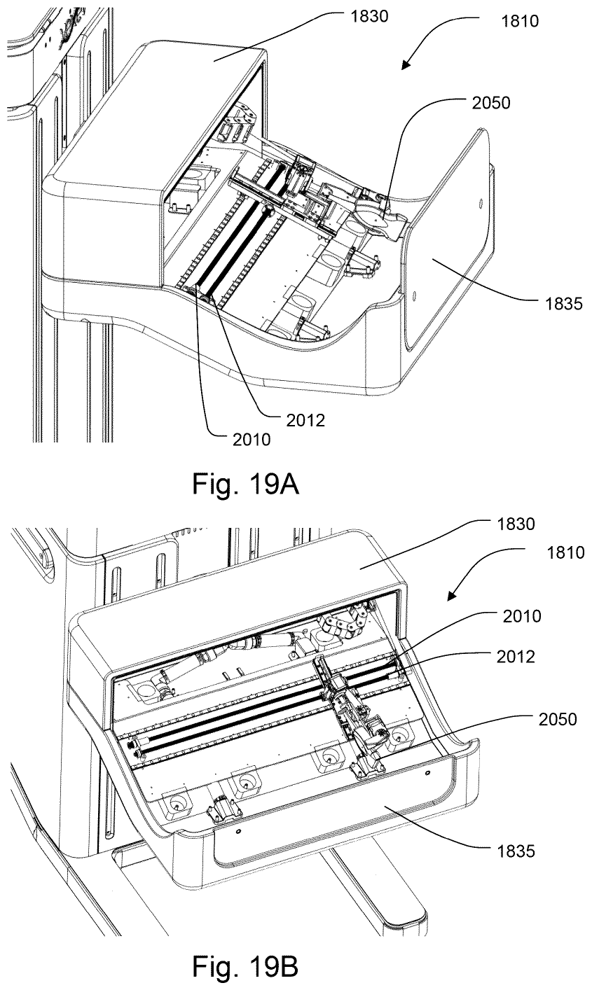

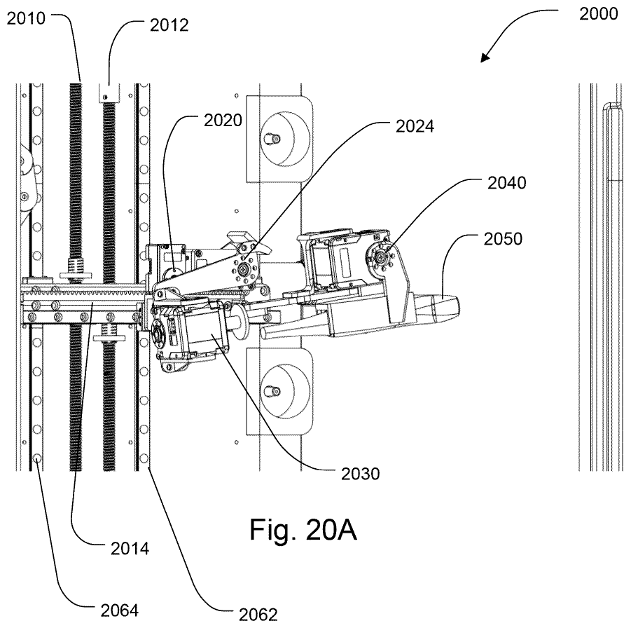

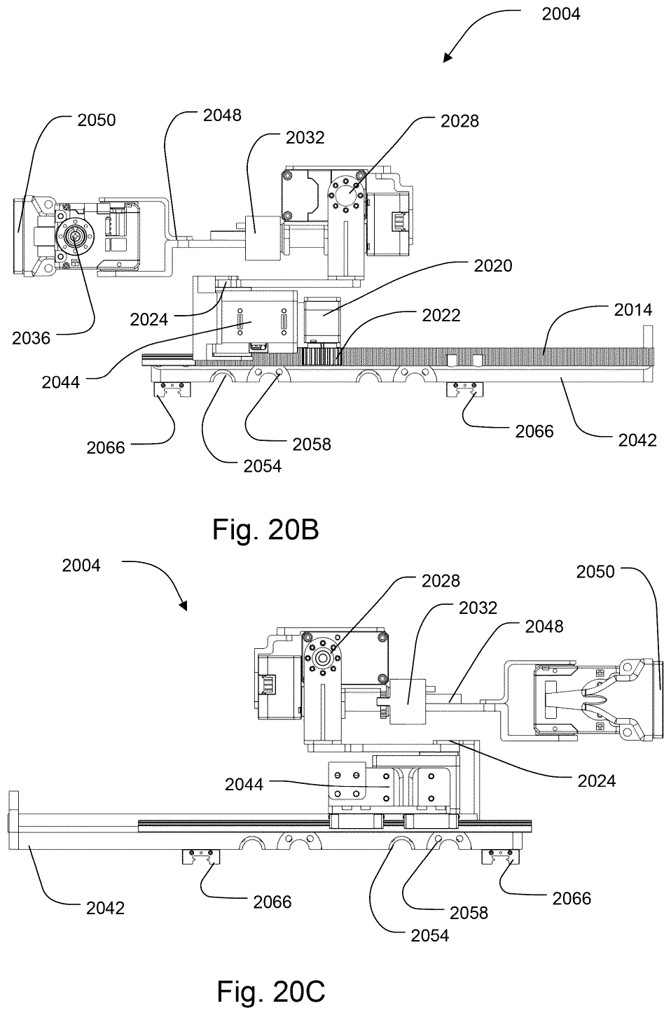

Another alternative single probe transport mechanism will now be described in relation to FIGS. 19A, 19B, 20A and 20B. The transport mechanism uses leadscrews instead of belts, along with other changes. However, the end result is similar, with an ultrasound probe being able to move to capture ultrasound images. FIGS. 19A and 19B show the adjustable scanning bed 1810, described above, with the flexible portion 1820 removed. FIGS. 20A and 20B show the transport mechanism in more detail.

A carriage is shown with three components, a carriage base 2042, a middle carriage 2044 and a upper carriage 2048. The carriage base 2042 moves along a single axis and is driven by a rear leadscrew 2010 and a front leadscrew 2012. The two leadscrews operate in tandem and keep the carriage base 2042 generally perpendicular to the leadscrews. A leadscrew nut on at least one of the rear leadscrew 2010 and/or front leadscrew 2012 is attached to a leadscrew attachment point 2058. Parts of the leadscrews without a leadscrew but pass under the carriage base 2042 through leadscrew clearance 2054. The carriage also has rail attachment points 2066 that connect the carriage to an inner rail 2062 and an outer rail 2064. Mounted on the carriage base 2042 is a rack 2014 and pinion 2022 that allow the middle carriage 2044 to move in a single direction, toward or away from the anatomic region of interest, driven by a pinion motor 2020. The upper carriage 2048 can move in a number of direction with motion about a upper carriage yaw rotation point 2024, roll rotation point 2032, driven by roll motor 2030, ultrasound probe rotation point 2036 and ultrasound probe yaw rotation point 2040. Each of the rotation points allows a motor to move a portion of the upper carriage 2048 in one degree of freedom. The result is that an ultrasound probe 2050 can be moved in six degrees of freedom.

### Dual Probe Transport Mechanism for a Robotic Ultrasound System

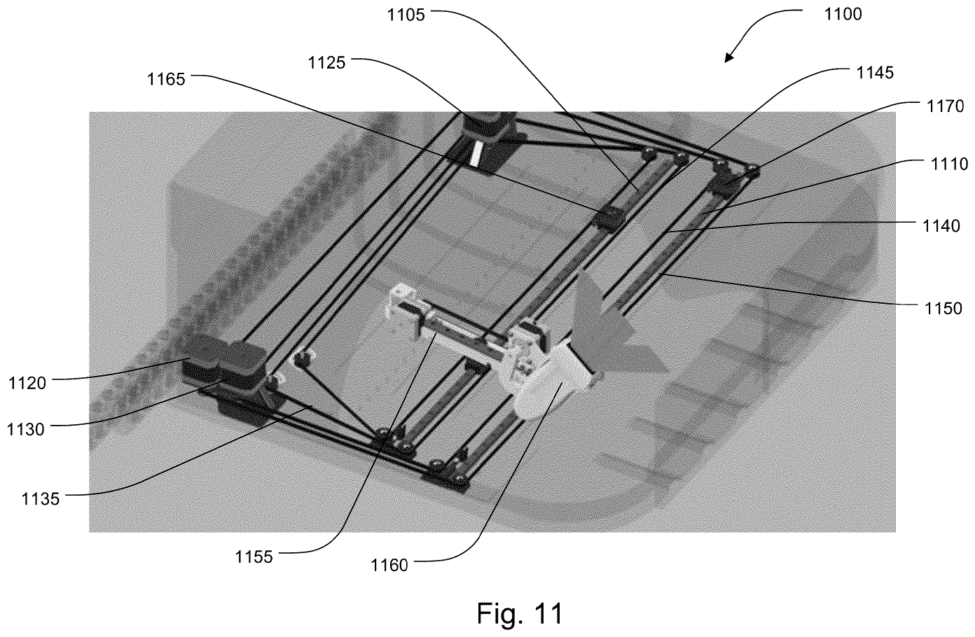

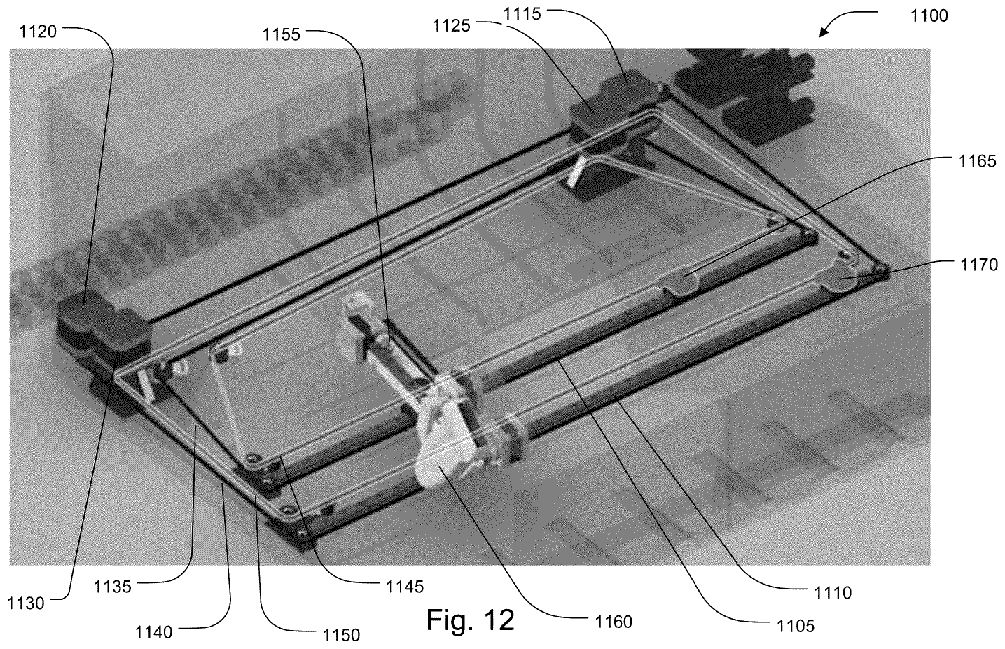

A dual probe robotic ultrasound system will now be described in relation to FIG. 11 and FIG. 12. The dual probe robotic ultrasound system is similar to the single probe robotic ultrasound system described above but provides a second ultrasound probe for additional ultrasound imaging capacity. Each of the probes of the dual probe robotic ultrasound system are submerged in the fluid of the fluid filled portion of the robotic ultrasound system.

In one embodiment, a dual probe transport 1100 uses a dual rail configuration as used in the transport mechanism 900 with an outer rail 1105 and an inner rail 1110 located closer to an anatomic region of interest. As with the transport mechanism 900, dual probe transport 1100 may also be located in the fluid filled portion. The arrangement of the dual probe transport 1100 provides for two ultrasound probes with the probe operating on a left and a right hand side of the outer rail 1105 and the inner rail 1110. Drive of a left hand side ultrasound probe 1160 is provided by a left probe outer rail motor 1115 and a left probe inner rail motor 1120. The motors of the left probe 1160 are connected to a left probe carriage 1155 on which the left probe 1160 is mounted. A left probe outer rail belt 1135 and a left probe inner rail belt 1140 connect the left probe outer rail motor 1115 and the left probe inner rail motor 1120 and allow the motors to traverse and slew the left probe carriage 1155. Movement of the left probe 1160 on the left probe carriage 1155 may be carried out in a similar manner as described above for the carriage 935.

A right side ultrasound probe, not shown, may be mounted on a right probe carriage, also not shown. Typically the left probe carriage 1155 and the right probe carriage will have matching design and operation. The right probe carriage moves along the outer rail 1105 and the inner rail 1110 using a right probe outer rail motor 1125, driving a right probe outer rail belt 1145, and a right probe inner rail motor 1130 driving a right probe inner rail belt 1150. A right probe outer rail carriage mount 1165 and a right probe inner rail carriage mount 1170 connect to the right probe outer rail belt 1145 and the right probe inner rail belt 1150, respectively, are attached to the right probe carriage.

As the left probe carriage 1155 and the right probe carriage are both attached and move along the outer rail 1105 and the inner rail 1110 the two carriages are unable to pass each other during operation of the dual probe transport 1100. Position tracking may be used to implement collision prevention to maintain separation between the two carriages and may take into account the position and slew of each of the carriages.

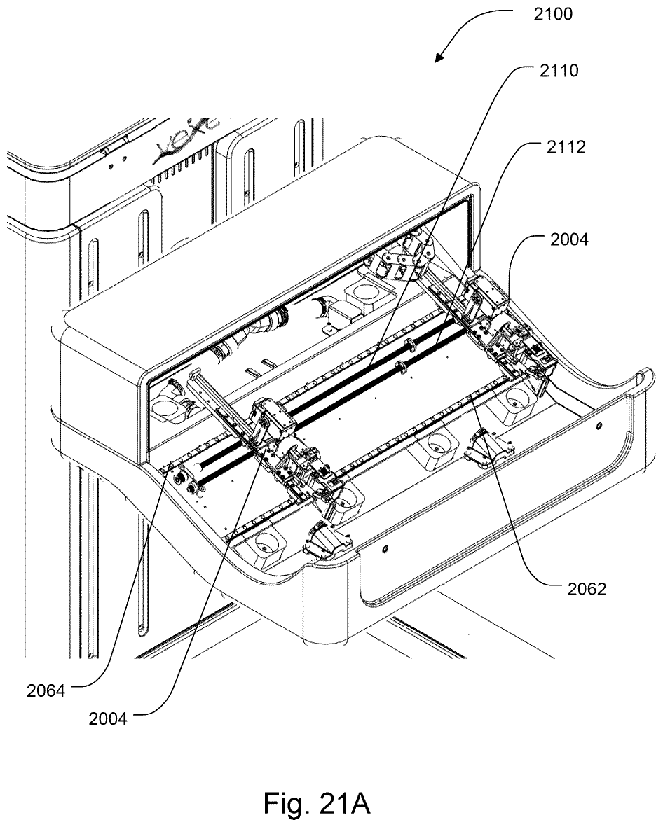

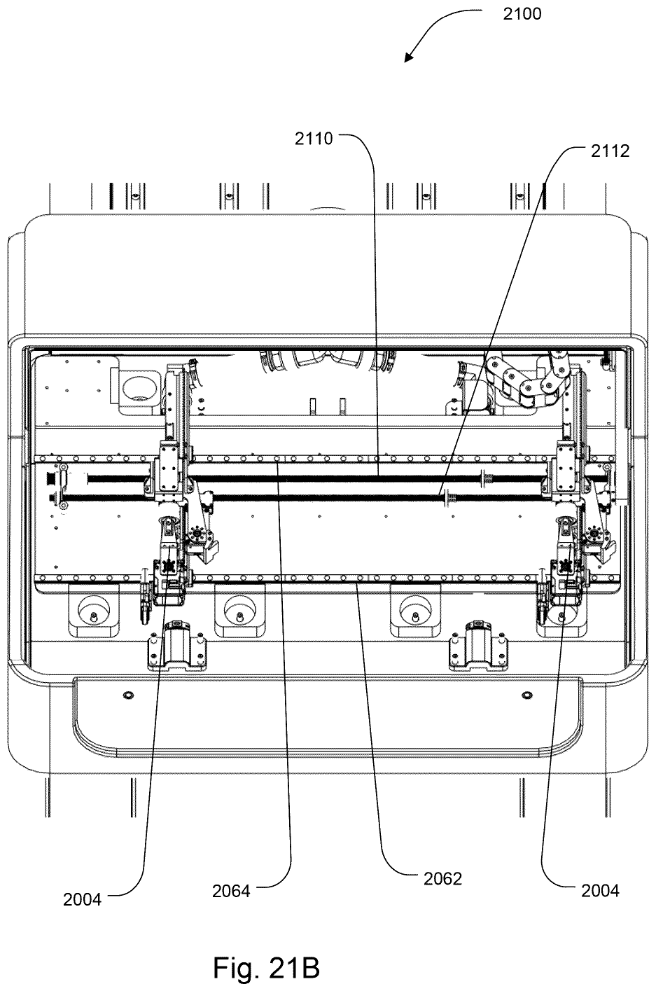

An alternative dual probe robotic ultrasound system will now be described in relation to FIGS. 21A and 21B. Shown is a scanning bed 2100 with the flexible portion removed. Mounted on the inner rail 2062 and the outer rail 2064, and moved by rear leadscrew 2010 and front leadscrew 2012, are carriages 2004. The carriage 2004 may be the carriages as described in relation to FIGS. 20A, 20B and 20C above. The carriage 2004 may be used in single or dual probe configurations of the robotic ultrasound system. When used in a dual probe configuration, one leadscrew, such as the rear leadscrew 2010, may move a left carriage 2004 and the other leadscrew, such as the front leadscrew 2012, may move the right carriage 2004.

### Scanning Method

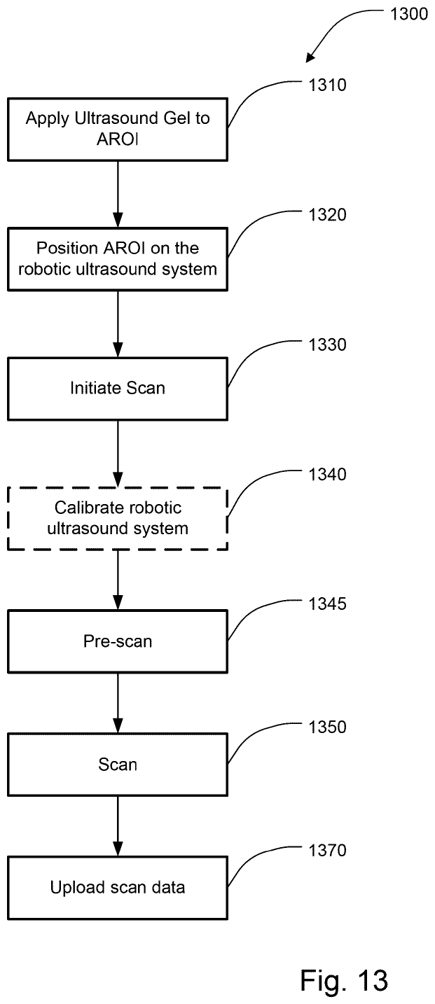

A robotic ultrasound system scanning method 1300 will now be described in relation to FIG. 13. The robotic ultrasound system scanning method 1300 describes how the robotic ultrasound system may be used to scan an anatomic region of interest of a patient. First, a prepare scanning region step 1310 takes place where the anatomic region of interest of a patient is prepared. Preparation may include application of ultrasound, aqueous gel to the anatomic region of interest and/or the flexible portion, before being placed on the flexible portion of the robotic ultrasound system.

Next, at a position anatomic region of interest step 1320 the anatomic region of interest is placed on the flexible portion of the robotic ultrasound system, facing towards a scanning window of the robotic ultrasound system, such as the scan window 330 of the robotic ultrasound system 300. The robotic ultrasound system may be situated on a height adjustable trolley or platform, or may be inbuilt into an arm-rest of a chair, table, hospital bed etc. The anatomic segment may be comfortably positioned on the flexible portion. Different arrangements may be made depending which anatomic region of interest is being scanned. For example, in the case an arm being scanned, the patient may hold an object and/or may use an ergonomic support such that the arm movement is limited with no degrees of freedom for the arm to be positioned in any other position. Similarly, when a leg is being scanned, the leg may be braced in a similar ergonomic manner.

At an initiate scan step 1330 a scan of the anatomic region of interest is initiated on the robotic ultrasound system controller software. The robotic ultrasound system controller software may be executed on a cloud based controller or may be executed on a local controller as described for the robotic ultrasound system controller software 1420. When the scan is initiated, a user of the robotic ultrasound system controller software may enter information such as type of scan, patient, clinic and device. In some examples, the robotic ultrasound system may automatically identify the patient from a biometric scanner instead of having the information manually entered. The robotic ultrasound system controller software may issue a unique identifier for the confidential patient data to be stored under or mapped to.

At a calibrate robotic ultrasound system step 1340 an optional calibration of the robotic ultrasound system may be performed. The calibration may be related to the transport system, such as positioning any carriages, such as the carriage 935, against limit switches to reset position encoders for the drive motors. In one example the ultrasound probe may perform a calibration operation, such as scanning a known calibration object contained within the fluid filled portion of the robotic ultrasound system, as described above.

A pre-scan step 1345 performs an initial scan of the anatomic region of interest to determine where the skin and other parts of the anatomy are located and to check the anatomic region of interest has been place in the correct location for scanning. The scanning that follows may be conducted in a sequential or non-sequential manner where different parts of the scanning are done, such as scanning at different angles. The output of the pre-scan step 1345 may be geometrical attributes that can be used during ultrasound scanning. Temporally varying flow measurement may also be collected, for anywhere in the anatomic region of interest. At a scan step 1350 scan information is collected. The scan step 1350 may perform different scan types, as selected by a user. The scan type may be pre-mapping, AV fistula, monitoring, tissue, muscle, or cancer. In one embodiment, at the scan step 1350, ECG-gated scan slices are obtained for the length of the total scannable volume. The scans are triggered during a predetermined part of the cardiac cycle using ECG information. Alternatively, the scans can be triggered by a pulse oximeter. The scan slices are accumulated in storages, such as memory of the robotic ultrasound system controller. The scan information may be stored in a raw format, such as raw RF format, or may be stored in a compressed format.

During the scan step 1350 the robotic ultrasound system may collect temporally varying flow measurements for end slices to fully define boundaries of the ultrasound scan. Finally, at an upload scan data step 1370 the scan data collected during operation of the robotic ultrasound system scanning method 1300 may be uploaded to a computer where the scan information may be processed. In one example, the scan information may be compressed before sending to a remote server for processing. The scan information may be sent over the network to a remote processor or the scan information may be processed locally. Although the upload scan data step 1370 is shown occurring after completion of the ultrasound scan, the scan information may commence being uploaded/transferred, to a local or remote server, once scans are collected at the scan step 1350. Data may continue to be uploaded as a parallel process to collection of further scan information. Alternatively, all the scan information may be collected before being sent for processing. In one example, the data may be processed in real time to provide feedback during the ultrasound scanning. The feedback may be used by the ultrasound system to make dynamic changes to the ultrasound scanning process.

Once the robotic ultrasound system scanning method 1300 sends the scan data and the scan data has been processed, the processed information may be used for diagnostic purposes.

### Robotic Ultrasound System Operation and Maintenance

To conduct a scan using the ultrasound system, the system is turned on and powered by either mains power or a battery. To prepare for scanning, the scanning bed is filled with fluid and the fluid is heated. Am ultrasound system that used regularly may have the fluid heated before first operation, with the heating system starting based on a timer. The ultrasound system may have an indicator to show that the system has reach a set temperature. An alarm may sound if a scan is initiated before the fluid is at the correct temperature. A scan type, such as pre-mapping, AV fistula, or monitoring, is selected using a suitable user interface. A user of the ultrasound system can also check the patient details with the user interface.

Before scanning, the scan area is checked for sharp objects, the system is located in position and the scanning bed is set at a correct level and angle. Gel is applied to the anatomic region of interest of the patient and/or the scanning bed, before placing the anatomic region of interest on the scanning bed. The operator of the ultrasound system may detach a touch screen on the system, if desired. Once the scan type is selected, the patient is prepared and position. Once the system is ready the scan may start.

A pre-map operation can show a potential scanning error, such as the anatomic region of interest is incorrectly positioned. If the patient moves during scanning, the ultrasound system may alert the operator and the scan restarted. Completion of the scan may be indicated to the operator and a scan successful or scan error message displayed. After the scan is completed, the patient removes the anatomic region of interest, the gel is removed from the patient and the ultrasound system and other suitable cleaning is performed.

The results from the ultrasound system scan can be viewed on the display on the system or another device, such as a tablet or computer through a suitable portal. The results may be used to read parameters on a display, label arteries/veins, obtain flow rate values, add labels to ultrasound images and/or make comments. Ultrasound clinical reports may also be created and exported from the system.

As described above, the ultrasound system has wheels that allows the system to be moved. Moving the system can be done after powering down the system, disconnecting any external power supplies and releasing a brake on the wheels. When moving the robotic ultrasound system for longer distances, such as moving using a vehicle transport, the fluid is drained from the system, the scanner bed stowed in a travel position or detached and the display screen secured or removed.

Maintenance of the robotic ultrasound system can be carried out at regular intervals, either based on a number of operating hours or time since the last maintenance. The fluid can be inspected through an access hatch on the tower. Tubes, pipes and pumps can be inspected for fluid leaks. Dust filters and fluid filters, located in the tower, may be cleaned. The scanning bed can be emptied of fluid and the top of the scanning bet removed to inspect the internal components, such as the motors, carriages, ultrasound scanner and cables. The ultrasound scanner can be examined to ensure that cables, housing lens and connectors are in good condition. The detachable screen can also be inspected to ensure that all user interface aspects are operational and the cable is in good condition. Software on the system may also be updated.

The ultrasound system should also be cleaned after use with any ultrasound gel removed and contact surfaces sanitised. The contact surfaces include the flexible portion of the scanning bed, housing of the trolley and touchscreen display.

### Variations

While motion on the carriage is described above as being driven by motors and belts, other arrangements may also be used. In one example, the belt may be replaced by a lead screw or a ball screw arrangement with the motors driving the screws and encoders attached to the screws determining position of the components of the carriage. Screw based drive systems may provide reduced backlash compared to a belt drive system.

In one example, the dual probe transport 1100 may use more than two rails. For example, a left probe carriage may be mounted on two separate rails to a right probe carriage. In one example, the carriages may be mounted on a single rail with a motor on the carriage providing rotation to slew the carriage about the rail. In some examples, such as the robotic ultrasound system 400, the range of motion of the ultrasound probe may be increased to allow scanning of curved flexible portions, such as the curved flexible portion 440. In dual ultrasound probe systems each ultrasound probe may be able to scan one of the curved flexible portions.

The examples described above show the scan window of the robotic ultrasound system located on one side of an anatomic region of interest of a patient to perform scans from one side and underneath the anatomic region of interest to enable scanning around the anatomic region of interest. In alternative system the scan window may be located underneath the patient with an ultrasound probe pointing up to the patient. Such an arrangement may allow for a narrower robotic ultrasound system with the probe transport, or transports, located under the patient. Such an arrangement may be useful where portability is important. In one alternative, the scan window may be located to one side of the anatomic region of interest. In another alternative, the scanning bed may be rotated 180 degrees so that ultrasound scanning can be conducted from the top of the anatomic region of interest.

In one alternative, there may be ultrasound probes scanning from a left side, a right side, underneath, above or some combination of the four directions. When scanning from underneath, or below, the ultrasound probe will face upwards to scan the anatomic region of interest. A robotic ultrasound system may have ultrasound probes located on either side to provide a more detailed view of the patient. The flexible portion of the shell of the robotic ultrasound system may also be larger to allow a neck or torso of a patient to be scanned. In one example, the robotic ultrasound system may be part of a bed, with a patient lying on a flexible portion, supported by the fluid in the robotic ultrasound system.

The robotic ultrasound system may determine fluid levels in the system using a level sensor to monitor a level of fluid in the system. In one embodiment, the ultrasound probe may act as a level sensor to determine fluid levels as the ultrasound probe can detect the location of the air above the fluid. If using a dedicated level sensor to determine fluid level, a level detecting system may use a series of discrete switches to monitor fluid levels or may use a system capable of providing a level measure. The fluid monitoring system may be used to ensure that the fluid level is suitable to cover the ultrasound probe as well as a path of the ultrasound waves. The fluid monitoring system may also be used to determine that the anatomic region of interest of the patient is located on the flexible portion of the robotic ultrasound system. As a limb of the patient is placed on the flexible portion, the fluid level in the robotic ultrasound system will rise. The change in the fluid level may be used to determine that presence and absence of the anatomic region of interest.

The fluid level in the scanning bed may be changed by the robotic ultrasound system to move the anatomic region of interest on the scanning bed up or down. In one embodiment the fluid level may be varied to allow the anatomic region of interest to be wrapped by the flexible portion. By lowering the fluid level, the anatomic region of interest may sink into the flexible portion and allow more regions of the anatomic region of interest to be scanned. The fluid is typically pumped to or from the reservoir to change the fluid level of the scanning bed.

The motors used for the robotic ultrasound system may be stepper motors. The stepper motors may be used in an open loop configuration or in a closed loop configuration with an encoder to determine the amount of movement. In one example, rotary encoders may be used on sprockets, such as the right rail sprocket 1005 and the middle sprocket 1010 of the carriage 935 or sprockets used for the outer rail belt 925 and the inner rail belt 930. An alternative is to use a servo motors which provides a closed loop configuration for the motors, or a combination of stepper motors and servo motors.

While the robotic ultrasound system described above uses a handheld ultrasound mounted on a transport mechanism, other ultrasound scanners may be used. Larger, or smaller, ultrasound scanners may be used that may be mounted on a carriage of the transport mechanism. The ultrasound probes used in the robotic ultrasound system may be capacitive or mechanical transducers such as micromachined ultrasonic transducers piezo-electric transducers, or any other type of mechanical transducer. The micromachined ultrasonic transducers may be CMUT (capacitive micromachined ultrasonic transducers) or PMUT (piezoelectric micromachined ultrasonic transducers), or a combination of both with CMUT and PMUT in one or more probes.

Examples of sound or acoustic waves include but are not limited to pressure waves, mechanical and longitudinal waves, and ultrasound waves. Ultrasound waves may comprise acoustic waves having a frequency greater than about 20 kHz. In some examples, the one or more mechanical transducers are ultrasound transducers configured to generate and detect ultrasound waves.

In some examples, the robotic ultrasound system comprises a single, or no more than one, mechanical transducer. In other examples, the robotic ultrasound system comprises a plurality of, such as two or more, mechanical transducers. The mechanical transducers of the plurality of mechanical transducers may be positioned or disposed in any arrangement or configuration. For example, two or more mechanical transducers may be arranged on a surface in a two-dimensional array or in a tiled configuration, such as a rectangular array, a square array, a circular array, or any other two or three-dimensional array shape.

Communications and power to the ultrasound probe may be delivered by USB, micro-coaxial, powered Ethernet, or an alternative means. In one alternative the power may be provided by a wired connection and the communication provided by a wireless communication protocol. The communication lines may also be used to update firmware of the ultrasound probe and sent via the robotic ultrasound system controller software. The robotic ultrasound system controller software may also be updated over a network connection to modify operation of the robotic ultrasound system. Such an operation may be used to update scanning processes of the robotic ultrasound system so that, for example, the robotic ultrasound system may be able to perform different diagnostic scans.

The ultrasound scan data collected by the robotic ultrasound system may be stored and processed either locally or on a remote server. In one example, the ultrasound scan data may be geometrical, 3 dimensional, scan data formed using locations of the ultrasound probe during scanning. The location may be tracked using the carriage and ultrasound probe position information from robotic ultrasound system controller. As described at the scan step 1350, the ultrasound scans of the robotic ultrasound system may have a timed capture according to the blood pulse cycle of a patient to account for variations in vein and/or artery changing in size as a heart of the patient beats. A trigger, such as electrocardiogram (ECG) may be used to select or trigger capture of data from the ultrasound probe or, alternatively, pulse oximetry gated acquisition may be used. The trigger may be used to determine a current stage of the stage of the cardiac pulse cycle and synchronise the date capture with the cardiac pulse cycle. In a further example, the ultrasound probe may capture blood flow rate, or blood flow velocity, for the anatomic region of interest. The ultrasound probe may be positioned by the robotic ultrasound system controller software to identify a particular vessel and obtain blood flow velocity. The ultrasound probe may be moved to more than one position to get an accurate measurement. As such, the samples may be aligned with a time domain, such as ECG. When the robotic ultrasound system uses more than one ultrasound probe, each of the ultrasound probes may require synchronisation using the ECG.

One example of a robotic ultrasound system may have processing in the robotic ultrasound system controller software to allow display of real-time images collected from the ultrasound probe. The robotic ultrasound system controller software may allow the operator to direct the ultrasound probe, or probes, to capture data based on the real-time images being displayed. Such an arrangement may be considered as semi-automated operation of the robotic ultrasound system.

As described above, the robotic ultrasound system may allow the patient to hold an object and/or may use an ergonomic support such that there is no degrees of freedom for an arm to be positioned. In the case where a leg of the patient is being scanned, the leg may be braced in a similar ergonomic manner. Sensors may be incorporated into the brace or held object. The sensors may be incorporated as part of the brace or held object or the sensors may be attached externally. The sensors may also be built in, or attached, to the flexible portion of the robotic ultrasound system. The sensor may include additional ultrasound units, temperature sensors, infrared sensors, lasers, fingerprint scanners, pressure sensors, cameras, etc and may be configured to provide pulse information, ECG data, movement information or other patient information. The sensors may also provide biometric information to identify the patient. In one example, pressure sensors may be used to determine if the patient modifies their grip on a hand hold or moves a limb.

While the robotic ultrasound system described above uses electric motor in a fluid such as mineral oil, other fluids may be used on the robotic ultrasound system. In one example, the robotic ultrasound system may be filled with water as the water will conduct the ultrasound waves from the ultrasound probe. However, as water is conductive, the electronics located in the fluid may be moved or use waterproof components. In one example, the movement of the transport mechanism and the carriage may be driven mechanically with the motors positioned separate to the water. Alternatively, the transport mechanism and the carriage may be moved using a hydraulic system. Any fluid used by the robotic ultrasound system may be drained from the system using a suitably positioned drainage tap.

The transport mechanism described physically positions the ultrasound probe to conduct a scan. In one example, the transport mechanism and carriage may be replaced with components that provide limited movement or no movement and use a 2D CMUT or PMUT array.

While the robotic ultrasound system has been described for the use on human patients, the robotic ultrasound system may also be used to scan any objects that can be imaged by ultrasound.

The robotic ultrasound system described above has a housing and flexible portion. In some examples of the robotic ultrasound system, the housing may be reduced or even eliminated. In one example, the robotic ultrasound system is made entirely of flexible material of the flexible portion. That is, there is no separate housing. The fluid filled portion of the robotic ultrasound system is located within the flexible portion.

### Advantages and Interpretation

The robotic ultrasound system described above provides a number of advantages. One advantage is that the robotic ultrasound system can perform automated ultrasound scanning without the need for specialised operators, such as sonographers. The robotic ultrasound system also provides comfortable, quick and reliable 3D ultrasound imaging at low cost.

Another advantage of the robotic ultrasound system is that the system allows for longitudinal monitoring because of the fixed position for the tissue, limb or body so that each scan is standardised. This may be useful for monitoring vascular disease. As described, the anatomic region of interest may be placed on the flexible portion of the robotic ultrasound system using aids to ensure the anatomic region of interest is in a set position. If the anatomic region of interest is repeatedly placed on the flexible portion using position aids then the ultrasound probe will scan the anatomic region of interest from the same angle for each ultrasound scan. Such a setup may provide for a high degree of repeatability compared to a traditional ultrasound system where the positioning of the ultrasound probe is conducted by the ultrasound system operator.

The use of a robotic ultrasound system may also be effective at providing remote or telehealth scanning. The scan can be conducted and the scan data uploaded to remote servers that can allow specialists to review the information at remote locations.

The robotic ultrasound system may also provide a more in depth view and resolution of the vasculature, compared to a traditional ultrasound, due to non real-time scanning that allows post-processing of the raw data. The robotic ultrasound system can also implement a two way iteration of scanning where the signals are sent and received and the ultrasound scanning is updated based on the anatomy being scanned. The robotic ultrasound system may also automatically capture the blood flow rates and be able to analyse the blood flow using computational fluid dynamics.

The design of the transport mechanism, such as the transport mechanism 900 provides a low profile design that is able to capture ultrasound images with a wide range of angles while fitting into a small volume within the robotic ultrasound system.

As the robotic ultrasound system is able to perform ultrasound scans under control of the robotic ultrasound system controller, updates or modifications to the system may occur by updating or modifying the software of the robotic ultrasound system controller. For example, an updated scanning process may be downloaded to update an existing scanning process. Alternatively, a library of scanning operations may form part of the robotic ultrasound system controller software or be accessible over a network connection and downloaded to the robotic ultrasound system to configure the robotic ultrasound system controller.

One advantage of the robotic ultrasound system that uses heated fluid is that vessels of the patient are heated to a consistent temperature. Heating the vessels to a suitable temperatures can allow for increased venous and arterial dilation. The use of heated fluid may also allow for consistent measurements during scans made over time, regardless of room temperature, as a constant temperature of an anatomic region, from the heated fluid, of interest can produce consistent scan information.

Another advantage of the robotic ultrasound system is that unskilled staff may operate the system. The workflow load of operating the robotic ultrasound system may be compared to that of measuring blood pressure with an automatic sphygmomanometer.

The reference in this specification to any prior publication (or information derived from the prior publication), or to any matter which is known, is not, and should not be taken as an acknowledgment or admission or any form of suggestion that the prior publication (or information derived from the prior publication) or known matter forms part of the common general knowledge in the field of endeavour to which this specification relates.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

1. A robotic ultrasound system comprising:
an ultrasound probe;
a transport mechanism having a plurality of motors for moving the ultrasound probe in at least one direction; and
a scanning bed comprising the transport mechanism and the ultrasound probe, the scanning bed having a fluid filled portion containing the plurality of motors and the ultrasound probe, wherein a fluid of the fluid filled portion allows transmission of ultrasound waves from the ultrasound probe to a surface of the robotic ultrasound system.

2. The robotic ultrasound system according to claim 1, wherein the scanning bed of the robotic ultrasound system further comprises:
a housing; and
a flexible portion on which an anatomic region of interest of a patient may be placed for scanning by the robotic ultrasound system, the flexible portion being in contact with the fluid of the fluid filled portion.

3. The robotic ultrasound system according to claim 2, wherein the robotic ultrasound system further comprises:
a wall to support the flexible portion.

4. The robotic ultrasound system according to claim 3, wherein the wall is removable.

5. The robotic ultrasound system according to claim 4, wherein the robotic ultrasound system further comprises a level sensor to monitor a level of the fluid.

6. The robotic ultrasound system according to claim 5, wherein the level sensor is the ultrasound probe.

7. The robotic ultrasound system according to claim 5, wherein the level sensor is used to determine a presence of the anatomic region of interest on the flexible portion.

8. (canceled)

9. (canceled)

10. The robotic ultrasound system according to claim 9, wherein the transport mechanism for moving the ultrasound probe is located in the fluid filled portion.

11. The robotic ultrasound system according to claim 1, wherein the transport mechanism for moving the ultrasound probe provides movement of the ultrasound probe in a direction selected from a set of directions comprising lateral, longitudinal, vertical, pitch, roll, and yaw.

12. The robotic ultrasound system according to claim 1, wherein the ultrasound probe is submerged in the fluid of the fluid filled portion.

13. The robotic ultrasound system according to claim 1, wherein the scanning bed of the robotic ultrasound system further comprises:
a second ultrasound probe submerged in the fluid of the fluid filled portion; and
a second transport mechanism for moving the second ultrasound probe in at least one direction.

14. The robotic ultrasound system according to claim 11, wherein the first and the second transport mechanisms travel on an inner and an outer rail.

15. (canceled)

16. The robotic ultrasound system according to claim 1, wherein the fluid filled portion contains a calibration object for calibrating the ultrasound probe.

17. The robotic ultrasound system according to claim 1, wherein the fluid of the fluid filled portion is pressurised.

18. (canceled)

19. The robotic ultrasound system according to claim 1, wherein the fluid of the fluid filled portion is heated to increase venous and arterial dilation.

20. (canceled)

21. The robotic ultrasound system according to claim 2, wherein the robotic ultrasound system further comprises:
a tower to which the scanning bed is attached.

22. The robotic ultrasound system according to claim 16, wherein the scanning bed is height adjustable by moving relative to the tower.

23. (canceled)

24. (canceled)

25. The robotic ultrasound system according to claim 1, wherein the transport mechanism moves the ultrasound probe with six degrees of freedom within the fluid filled portion.

26. A method of performing an ultrasound scan using a robotic ultrasound system, the method comprising:
initiating a scan of an anatomic region of interest from a robotic ultrasound system controller of a robotic ultrasound system comprising:
an ultrasound probe;
a transport mechanism having a plurality of motors for moving the ultrasound probe in at least one direction; and
a scanning bed comprising the transport mechanism and the ultrasound probe, the scanning bed having a fluid filled portion containing the plurality of motors and the ultrasound probe, wherein a fluid of the fluid filled portion allows transmission of ultrasound waves from the ultrasound probe to a surface of the robotic ultrasound system, the scanning bed further comprising a housing and a flexible portion on which an anatomic region of interest of a patient may be placed for scanning by the robotic ultrasound system, the flexible portion being in contact with the fluid of the fluid filled portion;
calibrating the robotic ultrasound system; and
collecting data for a length of a scannable volume.

27. The method according to claim 26, wherein calibrating the robotic ultrasound system comprises capturing scan data of the calibration object within the fluid filled portion.
